(19) United States
(12) Patent Application Publication
(10) Pub. No.: US 2025/0367218 A1
(43) Pub. Date: Dec. 4, 2025

(54) METHODS OF TREATING METABOLIC DISORDERS AND COMBINATION PRODUCTS FOR USE IN THE SAME

(71) Applicants: Carlos Batthyány, Montevideo (UY); INSTITUT PASTEUR DE MONTEVIDEO, Montevideo (UY); UNIVERSIDAD DE LA REPÚBLICA, Montevideo (UY); MINOZAR S.A., Montevideo (UY)

(72) Inventors: Carlos José Escande Castro, Montevideo (UY); Gloria Virginia Lopez, Montevideo (UY); Carlos Ignacio Batthyány Dighiero, Montevideo (UY); Maria Pia Garat, Montevideo (UY); Karina Beatriz Cal Castillo, Montevideo (UY); Santiago Ruiz Perera, Montevideo (UY); Leonardo Santos, Montevideo (UY); Maria Valentina Perez Torrado, Montevideo (UY)

(21) Appl. No.: 19/208,467

(22) Filed: May 14, 2025

Related U.S. Application Data
(60) Provisional application No. 63/647,555, filed on May 14, 2024.

(51) Int. Cl. A61K 31/60; A61K 38/26; A61P 3/04

(52) U.S. Cl. CPC A61K 31/60; A61K 38/26; A61P 3/04

(57) ABSTRACT

The present invention is directed to methods of treating a metabolic disorder in a subject in need thereof comprising administering to the subject a therapeutically effective amount of a nitro-vinyl benzene compound and administering to the subject a therapeutically effective amount of a GLP-1 agonist or analog. The present invention is also directed to kits and/or pharmaceutical compositions suitable for practicing the claimed methods.

### CROSS REFERENCE TO RELATED APPLICATION

This application claims priority to U.S. Provisional Application No. 63/647,555, filed on May 14, 2024, which is incorporated herein by reference.

### FIELD OF THE INVENTION

The present invention relates inter alia to methods of co-administering a nitro-vinyl benzene compound as described herein and a glucagon-like peptide-1 (GLP-1) agonist and/or analog for the treatment and/or prevention of metabolic diseases, disorders and conditions, including obesity, type 2 diabetes, MASH (Metabolic dysfunction-associated steatohepatitis), non-alcoholic fatty liver disease, metabolic dysfunction-associated fatty liver disease, non-alcoholic steatohepatitis, and/or fibrosing non-alcoholic steatohepatitis in a mammal. The present invention is also directed to compositions comprising a nitro-vinyl benzene compound as described herein and a GLP-1 agonist or analog for use in treating a mammal.

### BACKGROUND OF THE INVENTION

While the approval of therapies including GLP-1 agonists is showing that the pharmaceutical treatment of obesity and allied comorbidities is a reality, many aspects of the treatment of obesity and its comorbidities present several challenges. In particular, it is becoming clear GLP-1 agonists come with unpleasant side effects, such as gastroparesis, sarcopenia, etc. Additionally, the ability to maintain body weight once normal BMI is achieved and treatment is reduced or stopped, is suboptimal with current GLP-1 agonist based therapies. What is needed are new methods of administering GLP-1 agonists that permit lower and/or less frequent administration, reduced side effects, or a combination thereof.

### BRIEF SUMMARY OF THE INVENTION

The inventors have developed an alternative or complementary therapeutic strategy to the central pharmacological effect of GLP-1 agonists comprising co-administration of an active pharmaceutical ingredient that peripherally stimulates catabolism of adipose tissue by shuttling of the excess of energy to heat production or non-shivering thermogenesis.

Two tissues are principally responsible for heat production in mammals, skeletal muscle (shivering thermogenesis) and brown adipose tissue. However, under prolonged need, white adipose tissue also assumes heat-production properties by acquiring a brown adipose phenotype; a process termed “browning” or “beiging”. The principle molecular mechanism driving heat production by beige adipose tissue has long been believed to be up-regulation of the mitochondrial uncoupling protein UCP-1. However, UCP-1 knockout mice, when acclimated slowly, become resistant to cold exposure suggesting alternative mechanisms of heat production to UCP-1. Recently, a creatine-driven heat production pathway was identified to be operative in beige adipose tissue and activated independently of UCP-1. This pathway involves cycles of phosphorylation/dephosphorylation of creatine in the mitochondria, where the ATP produced by oxidative phosphorylation is consumed by phosphorylation, and heat is liberated by dephosphorylation. Pharmacological activation of this heat-production pathway in white adipose and brown tissue presents considerable opportunity to identify novel pharmaceutical targets as well as novel molecules for addressing obesity and diminishing its comorbidities.

The present inventors recently showed that a novel synthetic molecule, 5-(2-nitroethenyl) salicylic acid (referred to herein as “SANA” and/or “compound (I)”) stimulates thermogenesis and beiging of white adipose tissue by activating creatine-dependent thermogenesis in mice under diet-induced obesity (“DIO”). For example, obese mice treated with SANA demonstrate: 1) resistance to fat accretion, 2) improved glucose intolerance, 3) absence of liver steatosis, and 4) improved response to cold challenge, accompanied by up-regulation of key regulators of the creatine-dependent thermogenesis pathway (see, e.g., WO2020058917A1 and/or US2023346728A1).

The present inventors have carried out a number of experiments and discovered that the co-administration of SANA with a GLP-1 agonist or analog is surprisingly effective in treating obesity. Specifically, the co-administration of SANA and a GLP-1 agonist or analog has synergistic effects, and provides several benefits including but not limited to enhanced weight reduction, improved basal glucose levels, improved glucose tolerance, and a combination thereof compared to either agent administered alone.

Moreover, the specific combination of SANA and a GLP-1 agonist or analog appears to be synergistically active, thus permitting a GLP-1 agonist or analog to be administered at a lower dose, and thereby avoiding side effects, enhancing weight loss reduction, and/or extending the period of weight loss and/or the maintenance thereof.

As a result of the observed synergistic effects, the present invention permits the therapeutically effective dose of either SANA and/or a GLP-1 agonist or analog to be reduced by 10% or more, 20% or more, 30% or more, or 40% or more compared to the normal dose of SANA and/or a GLP-1 agonist or analog. In some embodiments, the methods are therefore directed to methods of achieving weight loss in a subject in need thereof comprising administering SANA in a reduced therapeutically effective amount by administering a GLP-1 agonist or analog. Similarly, the present invention is directed to methods of achieving weight loss in a subject in need thereof comprising administering a GLP-1 agonist or analog in a reduced therapeutically effective amount by administering SANA. Thus, the present invention enables methods of reducing bodyweight and/or improving cardiometabolic factors in a subject in need thereof, whilst reducing the known side effects of GLP-1 agonists and analogs and/or reducing any side effects from a higher dose of SANA.

Further, in some embodiments the present invention is directed to methods of administering a therapeutically effective amount of SANA and administering a therapeutically effective amount of a GLP-1 agonist or analog to a subject in need thereof, wherein the subject experiences greater weight loss than would be achieved by administering the same therapeutically effective amount of SANA alone or the same therapeutically effective amount of the GLP-1 agonist or analog alone.

In some embodiments, the present invention comprises a method of administering SANA and a GLP-1 agonist or analog to treat and/or prevent obesity and/or glucose management, as well as obesity-related comorbidities including but not limited to insulin resistance, type 2 diabetes, metabolic dysfunction-associated fatty liver disease (MAFLD), and combinations thereof in a mammal in need thereof with enhanced efficacy compared to SANA or a GLP-1 agonist or analog administered alone.

Further, the present invention is directed to methods of reducing the dose of a GLP-1 agonist tr analog to a subject being administered a GLP-1 agonist or analog by co-administering SANA. For example, the dose of a GLP-1 agonist or analog can be reduced once a desired weight loss is achieved and/or due to undesirable side effects of the GLP-1 agonist or analog. Thus, the methods of the present invention include reducing or minimizing weight regain and/or a “rebound effect” in a subject following a reduction of the dose of a GLP-1 agonist or analog.

Further, the present invention is directed to methods of administering SANA as a replacement therapy for a GLP-1 agonist or analog in a subject that discontinues treatment with a GLP-1 agonist or analog once a desired weight loss is achieved and/or due to undesirable side effects of the GLP-1 agonist or analog. Thus, the methods of the present invention include reducing or minimizing the side effects associated with administering a GLP-1 agonist or analog and/or reducing or minimizing weight regain and/or a “rebound effect” in a subject following discontinuation of a GLP-1 agonist or analog.

In some embodiments, the present invention is directed to a composition comprising: a therapeutically effective amount of compound (I)

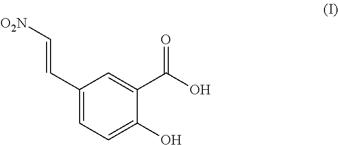 [SMILES: O=C(O)c1cc(/C=C/[N+](=O)[O-])ccc1O]

or a pharmaceutically acceptable salt thereof;
a therapeutically effective amount of a GLP-1 agonist or analog; and a pharmaceutically acceptable carrier.

In some embodiments, the GLP-1 agonist or analog is selected from: Albiglutide (Tanzeum), Aleniglipron (GSBR-1290), AMG133 (Maridebart cafraglutide, MariTide), Amycretin, AP025, AP026, ARI-2255, ARI-2651, Barnadutide (SAR425899), Beinaglutide, BGM0504 (BGM-0504, BGM 0504), 131-456906 (Survodutide), Birnagrurnab, Cagrilintide, CagriSema (Cagrilintide+Semaglutide), CAM-2056. Cinchonine, Cotadutide, CT-388 (RG6640), CT-868 (RG6641), CT-996 (RG6652), CVX-096, DA-15864, DA-3091 (microsphere formulation of exenatide), Danuglipron (PF-06882961), Dapiglutide (ZP7570), DD-01, DR10624, Dulaglutide (Trulicity), ECC5004, Ecnoglutide (XWV003 (Injectable), XW004 (Oral)), Efinopegdutide (HM12525A, MK-6024), Efocipegtrutide (HM15211), Efpeglenatide (LAPSExd4 analog, HM11260C), Exenatide (Byetta, Bydureon/Bydureon BCise), Exendin-4, GL0034 (Utreglutide), GLP-1, GLP-1 eligen, Glucagon, GMA106, GRMD-0901 (ORMD-0901), GSBR-1290 (Aleniglipron), GSK-2374697, HEC88473, HM15275, HRS-9531, HS-20094, 1Z010, HZ012, 1131362 (Mazdutide, OXM3), 1D110521156, Injectable HDV GLP1, Insulin glargine and lixisenatide (Soliqua 100/33, LixiLan), JYO9, Langlenatide, LAPSGlucagon Combo (HM14320), Liraglutide (Victoza, Saxenda), Lixisenatide (Adlyxin (US), Lyxumia (EU)), Lotiglipron (PF-07081532), Loxenatide, LY-2189265, LY-3305677, MAR-701, MAR709, Mazdutide (IBI362, OXM3), MDR-001, MET-097i, MK-8521, MKC-253, MOD-6030, MOD-6031, Noiiglutide (SH R20004, HS 20004), NN9423, NN-9709, NN-9924 (Oral Semaglutide), NN-9926, NN-9277, NNC0090-2746, NNC0487-0111, NNC0519-0130, OPK88003, Oral HDV GLP1, Orforglipron (LY3502970), ORM D-0901 (GRM D-0901), OWL-833, Oxyntomodulin, PB-1023, PB-718, Pegapamodutide, Pemvidutide (ALT-801), Petrelintide, PF-07081532 (Lotiglipron), PF-07976016, PYY 1875 (NNCO165-1875), Retatrutide (LY3437943, GGG Tri-agonist), rExendin-4, RGT-075, SAR425899 (Bamadutide), SAR441255, SCO-094, Semaglutide (Ozempic, Rybelsus, Wegovy), Septerna GLP-1R/GIPR/GCGR, SHR-1816, SHR20004 (Noiiglutide, HS 20004), Survodutide (BI-456906), Taspoglutide, TB001, TG103, Tirzepatide (Mounjaro, Zepbound), TT-401, TTP-054, TTP-273, UBT251, Viador-GLP-1, VK2735, Vurolenatide, XW014, ZP-2929, ZP-3022, ZP-DI-70, ZP5750, ZYD-1, ZYOG-1, polymer-bound analogs thereof, pegylated analogs thereof, pharmaceutically acceptable salts thereof, and combinations thereof. In some cases, the GLP-1 agonist or analog is semaglutide or a pharmaceutically acceptable salt thereof. In some cases, the GLP-1 agonist or analog is liraglutide or a pharmaceutically acceptable salt thereof.

In some embodiments, a composition as described herein is in the form of a solution, a suspension, a gel, an oil, a compressed tablet, an orally disintegrating tablet, a enteric coated tablet, a capsule, a pellet, a suppository, a powder, a lyophilized powder, an aerosol, an ointment, a cream, or a patch.

In some embodiments, compound (I) or a pharmaceutically acceptable salt thereof is present in an amount of about 0.1 mg to about 400 mg, and the semaglutide or a pharmaceutically acceptable salt thereof is present in an amount of about 0.25 mg to about 2.4 mg. In other embodiments, compound (I) or pharmaceutically acceptable salt thereof is present in an amount of about 0.1 mg to about 400 mg, and the semaglutide or a pharmaceutically acceptable salt thereof is present in an amount of about 3 mg to about 14 mg. In still other embodiments, compound (I) or pharmaceutically acceptable salt thereof is present in an amount of about 0.1 mg to about 400 mg, and the liraglutide or a pharmaceutically acceptable salt thereof is present in an amount of about 0.6 mg to about 1.8 mg.

In some embodiments, the present invention is directed to a kit comprising: a first composition comprising a therapeutically effective amount of compound (I)

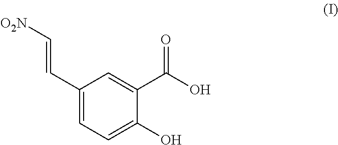 [SMILES: O=C(O)c1cc(/C=C/[N+](=O)[O-])ccc1O]

or a pharmaceutically acceptable salt thereof, and a pharmaceutically acceptable carrier; and a second composition comprising a therapeutically effective amount of GLP-1 agonist or analog, and a pharmaceutically acceptable carrier, wherein the first and second compositions are suitable for simultaneous or sequential administration to a subject in need thereof. The compositions and kits are suitable for practicing the methods described herein. In some embodiments, the first composition is present as one or more unit dosage forms suitable for oral or intravenous administration and the second composition is present as one or more unit dosage forms suitable for oral or intravenous administration. For example, in some embodiments, the first and second compositions are suitable for oral administration to a subject in need thereof. In some embodiments, the first composition is suitable for oral administration, and the second composition is suitable for injection to a subject in need thereof.

In some embodiments, in the kit, compound (I) or pharmaceutically acceptable salt thereof is present in an amount of about 1 mg to about 400 mg, and the GLP-1 agonist or analog is semaglutide or a pharmaceutically acceptable salt thereof present in an amount of about 3 mg to about 14 mg. In some embodiments, the kit comprises compound (I) or pharmaceutically acceptable salt thereof is present in an amount of about 1 mg to about 400 mg, and the GLP-1 agonist or analog is semaglutide or a pharmaceutically acceptable salt thereof present in an amount of about 0.25 mg to about 2.4 mg or liraglutide or a pharmaceutically acceptable salt thereof is present in an amount of about 0.6 mg to about 1.8 mg.

In some embodiments, the compositions, kits, and/or methods utilize compound (I) or a pharmaceutically acceptable salt thereof in an amount of about 0.1 mg to about 400 mg, and the semaglutide or a pharmaceutically acceptable salt thereof in an amount of about 0.25 mg to about 2.4 mg.

In some embodiments, the compositions, kits, and/or methods utilize compound (I) or a pharmaceutically acceptable salt thereof in an amount of about 0.1 mg to about 400 mg, and semaglutide or a pharmaceutically acceptable salt thereof in an amount of about 3 mg to about 14 mg.

In some embodiments, the compositions, kits, and/or methods utilize compound (I) or a pharmaceutically acceptable salt thereof is present in an amount of about 0.1 mg to about 400 mg, and the liraglutide or a pharmaceutically acceptable salt thereof is present in an amount of about 0.6 mg to about 1.8 mg.

In some embodiments, the kit may comprise a compound of compound (I) or pharmaceutically acceptable salt thereof present in an amount of about 1 mg to about 400 mg, and the GLP-1 agonist or analog is semaglutide or a pharmaceutically acceptable salt thereof present in an amount of about 0.25 mg to about 2.4 mg or liraglutide or a pharmaceutically acceptable salt thereof is present in an amount of about 0.6 mg to about 1.8 mg.

In some embodiments, the present invention is directed to a method of treating a disease or disorder in a subject in need thereof, comprising administering to the subject a therapeutically effective amount of compound (I)

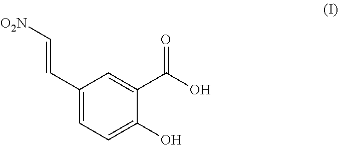 [SMILES: O=C(O)c1cc(/C=C/[N+](=O)[O-])ccc1O]

or a pharmaceutically acceptable salt thereof; and administering to the subject a therapeutically effective amount of GLP-1 agonist or analog, wherein the disease or disorder is selected from obesity, type 2 diabetes, MASH (Metabolic dysfunction-associated steatohepatitis), non-alcoholic fatty liver disease, metabolic dysfunction-associated fatty liver disease, non-alcoholic steatohepatitis, and/or fibrosing non-alcoholic steatohepatitis or a combination thereof in a mammal.

For example, in some embodiments, the subject in need thereof undergoes a reduction in body weight within 30 days of beginning treatment.

In some embodiments, the present invention is directed to a method of treating a disease or disorder in a subject being administered a therapeutically effective amount of GLP-1 agonist or analog, comprising determining the subject's current dose of GLP-1 agonist or analog; administering to the subject a therapeutically effective amount of compound (I)

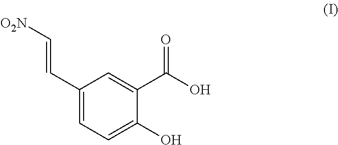 [SMILES: O=C(O)c1cc(/C=C/[N+](=O)[O-])ccc1O]

or a pharmaceutically acceptable salt thereof; and administering to the subject a lower dose of the GLP-1 agonist or analog, wherein the disease or disorder is selected from obesity, type 2 diabetes, MASH (Metabolic dysfunction-associated steatohepatitis), non-alcoholic fatty liver disease, metabolic dysfunction-associated fatty liver disease, non-alcoholic steatohepatitis, and/or fibrosing non-alcoholic steatohepatitis or a combination thereof in a mammal.

In some embodiments, at least one side effect or adverse effect associated with the GLP-1 agonist or analog is reduced in frequency, severity, or a combination thereof.

In some embodiments, the lower dose of the GLP-1 agonist or analog is at least 30% lower than the current dose, preferably at least 40% lower than the current dose, and most preferably at least 50% lower than the current dose.

In some embodiments, the present invention is directed to a method of reducing body weight, body fat, or a combination thereof in a subject in need thereof comprising: administering to the subject a therapeutically effective amount of a GLP-1 agonist or analog; and administering to the subject a therapeutically effective amount of compound (I) or a pharmaceutically acceptable salt thereof.

In some embodiments, the present invention is directed to a method of reducing and maintaining body weight, body fat, or a combination thereof in a subject in need thereof comprising: administering to the subject a therapeutically effective amount of a GLP-1 agonist or analog; and administering to the subject a therapeutically effective amount of compound (I) or a pharmaceutically acceptable salt thereof.

In some embodiments, in the methods described above, the GLP-1 agonist or analog is selected from: Albiglutide (Tanzeum), Aleniglipron (GSBR-1290), AMG133 (Maridebart cafraglutide, MariTide), Amycretin, AP025, AP026, ARI-2255, ARI-2651, Bamadutide (SAR425899), Beinaglutide, BGM0504 (3GM-0504, 13GM 0504), BI-456906 (Survodutide), Bimagrumab, Cagrilintide, CagriSema (Cagrilintide+Semaglutide), CAM-2056, Cinchonine, Cotadutide, CT-388 (RG6640), CT-868 (RG6641), CT-996 (RG6652), CVX-096, DA-15864, DA-3091 (microsphere formulation of exenatide), Danuglipron (PF-06882961), Dapiglutide (ZP7570), DD-01, DR10624, Dulaglutide (Trulicity), ECC5004, Ecnoglutide (XW003 (Injectable), XW004 (Oral)), Efinopegdutide (HM12525A, MK-6024), Efocipegtrutide (HM15211), Efpeglenatide (LAPSExd4 analog, HM11260C), Exenatide (Byetta, Bydureon/Bydureon BCise), Exendin-4, GL0034 (Utreglutide), GLP-1, GLP-1 eligen, Glucagon, GMA106, GRMD-0901 (ORMD-0901), GSBR-1290 (Aleniglipron), GSK-2374697, HEC88473, HM15275, HRS-9531, HS-20094, HZ010, HZ012, IBI362 (Mazdutide, OXM3), 11)110521156, Injectable HDV GLP1, Insulin glargine and lixisenatide (Soliqua 100/33, LixiLan), JYO9, Langlenatide, LAPSGlucagon Combo (HIM14320), Liraglutide (Victoza, Saxenda), Lixisenatide (Adlyxin (US), Lyxumia (EU)), Lotiglipron (PF-07081532), Loxenatide. LY-2189265, LY-3305677, MAR-701, MA R709, Mazdutide (IBI362, OXM3), MDR-001, MET-097i, MK-8521, MKC-253, MOD-6030, MOD-6031. Noiiglutide (SHR20004. HS 20004), NN9423, NN-9709, NN-9924 (Oral Semaglutide), NN-9926, NN-9277, NNC0090-2746, NNC0487-0111, NNC0519-0130, OPK88003, Oral HDV GLP1, Orforglipron (LY3502970), OR MD-0901 (GRMD-0901), OWL-833, Oxyntomodulin, PB-1023, PB-718, Pegapamodutide, Pemvidutide (ALT-801), Petrelintide, PF-07081532 (Lotiglipron), PF-07976016, PYY 1875 (NNC0165-1875), Retatrutide (LY3437943, GGG Tri-agonist), rExendin-4, RGT-075, SAR425899 (Bamadutide), SAR441255, SCO-094, Semaglutide (Ozempic, Rybelsus, Wegovy), Septerna GLP-1R/GIPR/GCGR, SHR-1816, SHR20004 (Noiiglutide, HS 20004), Survodutide (BI-456906), Taspoglutide, TB001, TG103, Tirzepatide (Mounjaro, Zepbound), TT-401, TTP-054, TTP-273, UBT251, Viador-GLP-1, V1K2735, Vurolenatide, XW014, ZP-2929, ZP-3022, ZP-DI-70, ZP5750, ZYD-1. ZYOG-1, polymer-bound analogs thereof, pegylated analogs thereof, pharmaceutically acceptable salts thereof, and combinations thereof.

In some embodiments, the subject in need thereof experiences a reduction of body weight within the first 30 days of administering the therapeutically effective amount of compound (I) or a pharmaceutically acceptable salt thereof.

In some embodiments, the present invention is directed to a method of maintaining body weight, body fat, or a combination thereof in a subject in need thereof currently taking a GLP-1 agonist or analog comprising: reducing the dose of the GLP-1 agonist or analog; and administering to the subject a therapeutically effective amount of compound (I) or a pharmaceutically acceptable salt thereof.

In some embodiments, the present invention is directed to a method of preventing weight gain in a subject currently taking a GLP-1 agonist or analog comprising: administering to the subject a therapeutically effective amount of compound (I) or a pharmaceutically acceptable salt thereof to the subject. In some embodiments, the method further comprises discontinuing the GLP-1 agonist or analog.

In some embodiments, the present invention is directed to a method of slowing the progression of Type-II diabetes in a subject in need thereof comprising: administering to the subject a therapeutically effective amount of a GLP-1 agonist or analog; and administering to the subject a therapeutically effective amount of compound (I) or a pharmaceutically acceptable salt thereof.

In some embodiments, the subject in need of treatment is obese, suffers from diabetes, suffers from Type-II diabetes, or a combination thereof.

In some embodiments, the methods of the present invention comprise administering of the GLP-1 agonist or analog prior to administering of the compound (I) or pharmaceutically acceptable salt thereof.

In some embodiments, the subject in need is a mammal. In some embodiments, the subject in need is a human.

### BRIEF DESCRIPTION OF THE FIGURES AND DRAWINGS

FIGS. 1A and 1B provide graphic representations of the observed weight of mice fed a high-fat diet (HFD) for 5.5 weeks previous to start treatments (Time 0). At that point mice were untreated (HFD), treated with SANA alone (HFD+SANA), treated with liraglutide alone (HFD+LIRA), or treated with concomitant administration of SANA and liraglutide (HFD+SANA+LIRA), with *, **, ***, and **** showing statistically significant differences, p<0.05, p>0.01, p<0.001, and p<0.0001, respectively.

FIG. 1C shows cumulative food intake in mice fed a high-fat diet (HFD) for 5.5 weeks and then were untreated (HFD), treated with SANA alone (HFD+SANA), treated with liraglutide alone (HFD+LIRA), or treated with concomitant administration of SANA and liraglutide (HFD+SANA+LIRA),

FIGS. 2A, 2B, and 2C provide graphic representations of glucose tolerance (FIG. 2A, GTT), basal glucose (FIG. 2B), and glucose area under the curve (AUC) (FIG. 2C) following treatment of mice with SANA alone, liraglutide alone, or a combination of SANA with liraglutide, with *, **, *** and **** showing statistically significant differences, p<0.05, p>0.01, p<0.001 and p<0.0001, respectively.

FIGS. 3A and 3B provide graphic representations of the observed weight of mice fed a high-fat diet (HFD) for 5.5 weeks previous to start treatments (Time 0). At that point mice were untreated (HFD), treated with SANA alone (SANA), treated with semaglutide alone (Semaglutide), or treated with concomitant administration of SANA and semaglutide (Semaglutide+SANA), with *, **, *** and **** showing statistically significant differences, p<0.05, p>0.01, p<0.001 and p<0.0001, respectively.

FIG. C shows X-ray images of mice fed a high-fat diet (HFD) that were untreated (HFD), treated with SANA alone (HFD+SANA), treated with semaglutide alone (HFD+SEMA), or treated with concomitant administration of SANA and semaglutide (HFD+SANA+SEMA).

FIG. 3D provides a graphic representation of cumulative food intake for untreated fed a high-fat diet (HFD) for 5.5 weeks previous to start treatments (Time 0). At that point mice were untreated (HFD), treated with semaglutide alone (HFD+SEMA), treated with SANA alone (HFD+SANA), or treated with concomitant administration of SANA and semaglutide (HFD+SANA+SEMA).

FIGS. 3E and 3F provide graphic representations of the observed weight of mice fed a high-fat diet (HFD) for 5.5 weeks previous to start treatments (Time 0). At that point mice were untreated (HFD), treated with 0.25 nmol/kg/day semaglutide alone, treated with 2 nmol/kg/day semaglutide alone, treated with SANA alone, treated with concomitant administration of SANA and 0.25 nmol/kg/day semaglutide, or treated with concomitant administration of SANA and 2 nmol/kg/day semaglutide.

FIG. 4 provides a graphic representation of the observed average weight of mice fed a high-fat diet for 5.5 weeks and then were treated with a semaglutide (HFD+SEMA, 40 mcg/kg subcutaneous injection administered every 3 days), SANA (HFD+SANA, 10 mg/kg/day, PO), concomitant administration of both semaglutide and SANA (HFD+SEMA+SANA), or no treatment (HFD (control)). * and ** show statistically significant differences, p<0.05 and p>0.01, respectively.

FIG. 5 provides a graphic representation of the observed average weight of mice fed a high-fat diet (HFD) for 5.5 weeks previous to start treatments (Time 0). At that point mice were untreated (HFD), treated with GLP-1 agonist (HFD+liraglutide, LIRA), or treated with SANA (HFD+SANA) for a period of 9 weeks, at which time treatment was discontinued and the weight regain of the mice was monitored for another 5 weeks. * shows statistically significant difference, p<0.05.

FIG. 6A provides a graphic representations of the observed average weight of mice fed a high-fat diet for 5.5 weeks and then were untreated (HFD), treated with GLP-1 agonist (semaglutide, 40 mcg/kg/3-days), or treated with semaglutide+SANA for a period of 2.5 weeks, at which time all groups were treated only with SANA (10 mg/kg/day, SC, daily administration) and the weight regain of the mice was monitored for another 1.5-6.5 weeks. * shows statistically significant difference, p<0.05.

FIGS. 6B and 6C provide graphic representations of the observed average weight of mice fed a high-fat diet for 5.5 weeks and then were untreated (HFD), treated with GLP-1 agonist (semaglutide, 10 nmol/kg/day—SC), or treated with semaglutide+SANA for a period of 1.5 weeks, at which time, semaglutide was ceased and groups were treated only with SANA (200 mg/kg/day, PO). The weight regain of the mice was monitored for another 1.5-6.5 weeks. *, **, ***, #, and ##show statistically significant differences, p<0.05, p<0.01, p<0.001, p<0.05, and p<0.01, respectively.

FIG. 6D provides a graphic representation of cumulative food intake for untreated fed a high-fat diet (HFD) for 5.5 weeks previous to start treatments (Time 0). At that point mice were untreated (HFD), or treated with concomitant administration of SANA and semaglutide (HFD+SANA+Semaglutide) for a period of 1.5 weeks, at which time semaglutide was stopped, though SANA was continued. Other mice were treated with semaglutide alone (HFD+Semaglutide 10 nmol/kg/day, subcutaneous injection administered every day), or treated with concomitant administration of SANA (200 mg/kg/day—PO) and semaglutide (HFD+SANA+Semaglutide) for a period of 1.5 weeks, at which time semaglutide was stopped though SANA was continued (HFD+SANA). The cumulative food intake for all mice was monitored for another 6.5 weeks.

FIGS. 6E and 6F provides a graphic representation of fasting glucose for mice fed a high-fat diet (HFD) for 5.5 weeks previous to start treatments (Time 0). At that point mice were untreated (HFD), treated with semaglutide alone (HFD+Semaglutide 10 nmol/Kg/day, subcutaneous injection administered every day) or treated with concomitant administration of SANA (200 mg/Kg/day—PO) and semaglutide (HFD+SANA+Semaglutide) for a period of 1.5 weeks, at which time semaglutide was stopped but not SANA (HFD+SANA). ** and *** show statistically significant differences, p>0.01 and p<0.001, respectively

FIG. 7A provides a graphic representation of cumulative food intake for mice fed a high-fat diet (HFD) for 5.5 weeks previous to start treatments (Time 0). At that point mice were untreated (HFD), treated with SANA alone, treated with 0.25 nmol/kg/day semaglutide alone, treated with 2 nmol/kg/day semaglutide alone, treated with concomitant administration of SEMA and 0.25 nmol/kg/day semglutide, or treated with concomitant administration of SANA and 2 nmol/kg/day semaglutide for a period of 4 weeks.

FIG. 7B shows a graphic representation of fasting glucose and serum non-esterified fatty acids (NEFA) at 4 weeks for untreated fed a high-fat diet that were untreated (HFD), treated with SANA alone, treated with 0.25 nmol/kg/day semaglutide alone, treated with 2 nmol/kg/day semaglutide alone, treated with concomitant administration of SANA and 0.25 nmol/kg/day semglutide, or treated with concomitant administration of SANA and 2 nmol/kg/day. semaglutide. * and ** show statistically significant differences, p<0.05 and p>0.01 respectively

FIGS. 8A and 8B provide graphic representations of the observed average weight of mice fed a high-fat diet for 5.5 weeks and then were untreated (HFD), treated with 50 mg/kg/day SANA alone, 100 mg/kg/day SANA alone, 200 mg/kg/day SANA alone, 2 nmol/kg/day semaglutide alone, concomitant administration of 50 mg/kg/day SANA and 2 nmol/kg/day semglutide, or concomitant administration of 100 mg/kg/day SANA and 2 nmol/kg/day semglutide, or concomitant administration of 200 mg/kg/day SANA and 2 nmol/kg/day semglutide, for a period of 1.5 weeks. *, **, *** and **** show statistically significant differences, p<0.05, p>0.01, p<0.001 and p<0.0001, respectively.

FIGS. 8C, 8D, and 8E show graphic representations of cumulative food intake, fasting glucose, and serum non-esterified fatty acids (NEFA) for mice fed a high-fat diet for 5.5 weeks, then untreated (HFD), treated with 50 mg/kg/day SANA alone, 100 mg/kg/day SANA alone, 200 mg/kg/day SANA alone, 2 nmol/kg/day semaglutide alone, concomitant administration of 50 mg/kg/day SANA and 2 nmol/kg/day semglutide, or concomitant administration of 100 mg/kg/day SANA and 2 nmol/kg/day semglutide, or concomitant administration of 200 mg/kg/day SANA and 2 nmol/kg/day semglutide, at the end of 1.5 weeks of treatment, for a period of 1.5 weeks. *, **, *** and **** show statistically significant differences, p<0.05, p>0.01, p<0.001 and p<0.0001, respectively.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meaning as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must also be noted that as used herein and in the appended claims, the singular forms “a,” “an,” and “the” include plural reference unless the context clearly dictates otherwise. Thus, for example, reference to a “cell” is a reference to one or more cells and equivalents thereof known to those skilled in the art, and so forth.

“Administering” when used in conjunction with a therapeutic means to administer a therapeutic directly to a subject, whereby the agent positively impacts the target.

“Administering” a composition may be accomplished by, for example, injection, oral administration, topical administration, or by these methods in combination with other known techniques. Such combination techniques include heating, radiation, ultrasound and the use of delivery agents. When a compound is provided in combination with one or more other active agents (e.g. other anti-atherosclerotic agents such as the class of statins), “administration” and its variants are each understood to include concurrent and sequential provision of the compound or salt and other agents.

As used herein, “pharmaceutically acceptable” includes any composition, material, excipient or combination of excipients suitable for it is meant the carrier, diluent, adjuvant, or excipient must be compatible with the other ingredients of the formulation and not deleterious to the recipient thereof.

By “combination therapy” or “polytherapy” it is meant therapy that uses more than one medication or modality (versus monotherapy, which is any therapy taken alone). Typically, these terms refer to using multiple therapies to treat a single disease, and often all the therapies are pharmaceutical (although it can also involve non-medical therapy, such as the combination of medications and talk therapy to treat depression). Pharmaceutical combination therapy may be achieved by prescribing/administering separate drugs, or, where available, dosage forms that contain more than one active ingredient (such as fixed-dose combinations). Polypharmacy is a related term, referring to the use of multiple medications (without regard to whether they are for the same or separate conditions/diseases). As used herein “polymedicine” is used to refer to pharmaceutical combination therapy. Most of these kinds of terms lack a universally consistent definition, so caution and clarification are often advisable.

“Composition” as used herein is intended to encompass a product comprising the specified ingredients in the specified amounts, as well as any product which results, directly or indirectly, from combination of the specified ingredients in the specified amounts. Such term in relation to “pharmaceutical composition” is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

As used herein, the term “agent,” “active agent,” “therapeutic agent,” or “therapeutic” means a compound or composition utilized to treat, combat, ameliorate, prevent or improve an unwanted condition or disease of a patient. Furthermore, the term “agent,” “active agent,” “therapeutic agent,” or “therapeutic” encompasses a combination of one or more of the compounds of the present invention.

A “therapeutically effective amount” or “effective amount” of a composition is a predetermined amount calculated to achieve the desired effect, i.e., to inhibit, block, or reverse the activation, migration, proliferation, alteration of cellular function, and to preserve the normal function of cells. The activity contemplated by the methods described herein includes both medical therapeutic and/or prophylactic treatment, as appropriate, and the compositions of the invention may be used to provide improvement in any of the conditions described. It is also contemplated that the compositions described herein may be administered to healthy subjects or individuals not exhibiting symptoms but who may be at risk of developing a particular disorder. The specific dose of a compound administered according to this invention to obtain therapeutic and/or prophylactic effects will, of course, be determined by the particular circumstances surrounding the case, including, for example, the compound administered, the route of administration, and the condition being treated. However, it will be understood that the chosen dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue.

The terms “treat,” “treated,” or “treating” as used herein refer to both therapeutic treatment and prophylactic or preventative measures, wherein the object is to prevent or slow down (lessen) an undesired physiological condition, disorder, or disease, or to obtain beneficial or desired clinical results. For the purposes of this invention, beneficial or desired results include, but are not limited to, alleviation of symptoms; diminishment of the extent of the condition, disorder, or disease; stabilization (i.e., not worsening) of the state of the condition, disorder, or disease; delay in onset or slowing of the progression of the condition, disorder, or disease; amelioration of the condition, disorder, or disease state; and remission (whether partial or total), whether detectable or undetectable, or enhancement or improvement of the condition, disorder, or disease. Treatment includes prolonging survival as compared to expected survival if not receiving treatment.

The term “subject,” as used herein, describes an organism, including mammals, to which treatment with the compositions and compounds according to the subject disclosure can be administered. Mammalian species that can benefit from the disclosed methods include, but are not limited to, apes, chimpanzees, orangutans, humans, monkeys; and other animals such as dogs, cats, horses, cattle, pigs, sheep, goats, chickens, mice, rats, guinea pigs, and hamsters. Typically, the subject is a human.

The term “tissue,” as used herein, describes an aggregate of cells typically of a particular kind together with their intercellular substance that form one of the structural materials of a subject. The term “organ,” as used herein, describes a group of tissues that perform a specific function. For example, skin is a type of organ embodied herein.

The compositions of the invention as described herein may be administered to subjects to treat obesity. In other embodiments, the compositions of the invention as described herein may be administered to subjects to prevent weight gain or reduce the rate of weight gain in a subject. In some embodiments, the compositions of the invention as described herein may be administered to induce weight loss in a subject. In some embodiments, the compositions of the invention as described herein may be administered to treat the subject for one or more adverse effects associated with weight gain and/or obesity, such as, but not limited to glucose intolerance. In some embodiments, the compositions of the invention as described herein may be administered to treat obesity. In some embodiments, the compositions of the invention as described herein may be administered to induce thermogenesis in a subject.

Advantageously, as further described herein, the presence of compound (I) in compositions, kits or methods of the invention, permits the reduction or halting of therapy with a GLP-1 agonist or analog to reduce or avoid side effects, whilst allowing weight loss or target weight maintenance to continue. In other words, the commonly observed gain of weight upon reducing or stopping GLP-1 agonist treatment can be avoided, reduced or ameliorated by switching to therapy, for example monotherapy, with compound (I).

The present invention utilizes a compound of Formula (I):

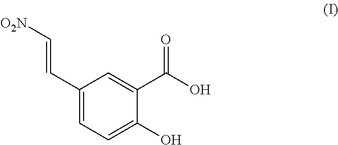 [SMILES: O=C(O)c1cc(/C=C/[N+](=O)[O-])ccc1O]

or a pharmaceutically acceptable salt thereof, which is also referred to herein as SANA or compound (I). In particular, the compositions, kits, and/or methods of the present invention utilize a therapeutically effective amount of compound (I) described herein.

Pharmaceutically acceptable salts of compound (I) include, but are not limited to base-addition salts, alkali salts, alkali earth metal salts, transition metal salts, with alkali salts and alkali earth metal salts being preferred.

As used herein, “GLP-1 agonists and analogs” includes GLP-1 and analogs thereof, agonists of the GLP-1 receptor (i.e., GLP-1 agonists), GLP-1 polyagonists, dual agonists of GLP-1 and glucagon receptors (i.e., dual GLP-1/Gcg agonists) peptide-2 (GLP-2) receptors (i.e., dual GLP-1/GLP-2 agonists), dual agonists of GLP-1 and glucose-dependent insulinotropic polypeptide (GIP) receptors (i.e., dual GLP-1/GIP agonists), dual GLP-1 agonist/GIP antagonists, GLP-1/Gcg/GIP tri-agonists, dual GLP-1/amylin agonists, dual GLP-1/fibroblast growth factor 21 (FGF21) agonists, GLP-1/Gcg/FGF21 tri-agonists, polymer-bound analogs thereof, pegylated analogs thereof, and combinations thereof.

GLP-1 agonists and analogs suitable for use with the methods and/or compositions of the present invention include, but are not limited to, Albiglutide (Tanzeum), Aleniglipron (GSBR-1290), AMG133 (Maridebart cafraglutide, MariTide), Amycretin, AP025, AP026, ARI-2255, ARI-2651, Bamadutide (SAR425899), Beinaglutide, BGM0504 (BGM-0504, BGM 0504), BI-456906 (Survodutide), Bimagrumab, Cagrilintide, CagriSema (Cagrilintide+Semaglutide), CAM-2056, Cinchonine, Cotadutide, CT-388 (RG6640), CT-868 (RG6641), CT-996 (RG6652), CVX-096, DA-15864, DA-3091 (microsphere formulation of exenatide), Danuglipron (PF-06882961), Dapiglutide (ZP7570), DD-01, DR10624, Dulaglutide (Trulicity), ECC5004, Ecnoglutide (XW003 (Injectable), XW004 (Oral)), Efinopegdutide (HM12525A, MK-6024), Efocipegtrutide (HM15211), Efpeglenatide (LAPSExd4 analog, HMI11260C), Exenatide (Byetta, Bydureon/Bydureon BCise), Exendin-4, GL0034 (Utreglutide), GLP-1, GLP-1 eligen, Glucagon, GMA106, GRMD-0901 (ORMD-0901), GSBR-1290 (Aleniglipron), GSK-2374697, HEC88473, HM15275, HRS-9531, HS-20094, HZ010, HZ012, IBI362 (Mazdutide, OXM3), ID110521156, Injectable HDV GLP1, Insulin glargine and lixisenatide (Soliqua 100/33, LixiLan), JY09, Langlenatide, LAPSGlucagon Combo (HM 14320), Liraglutide (Victoza, Saxenda), Lixisenatide (Adlyxin (US), Lyxumia (EU)), Lotiglipron (PF-07081532), Loxenatide, LY-2189265, LY-3305677, MAR-701, MAR709, Mazdutide (IBI362, OXM3), MDR-001, MET-097i, MK-8521, MKC-253. MOD-6030, MOD-6031, Noiiglutide (SHR20004, HS 20004), NN9423, NN-9709, NN-9924 (Oral Semaglutide), NN-9926, NN-9277, NNC0090-2746, NNC0487-0111, NNC0519-0130, OPK88003, Oral HDV GLP1, Orforglipron (LY3502970), ORMD-0901 (GRMD-0901), OWL-833, Oxyntomodulin, PB-1023, PB-718, Pegapamodutide, Pemvidutide (ALT-801). Petrelintide. PF-07081532 (Lotiglipron), PF-07976016, PYY 1875 (NNCO165-1875), Retatrutide (LY3437943, GGG Tri-agonist), rExendin-4, RGT-075. SAR425899 (Bamadutide), SAR441255, SCO-094, Semaglutide (Ozempic, Rybelsus, Wegovy), Septerna GLP-1R/GIPR/GCGR, SHR-1816, SHR20004 (Noiiglutide, HS 20004), Survodutide (BI-456906), Taspoglutide, TB001, TG103, Tirzepatide (Mounjaro, Zepbound), TT-401, TTP-054, TTP-273, UBT251, Viador-GLP-1, VK2735, Vurolenatide, XW014, ZP-2929, ZP-3022, ZP-DI-70, ZP5750, ZYD-1, ZYOG-1, polymer-bound analogs thereof, pegylated analogs thereof, and combinations thereof.

In some embodiments, the methods of the present invention and/or the compositions of the present invention further include administering SANA alone, in combination with a GLP-1 agonist or analog, and with a third active pharmaceutical ingredient having activity in treating cardiometabolic conditions, in particular obesity, selected from: a GLP-2 agonist, a Gcg agonist, a GIP agonist, an amylin receptor agonist, a neuropeptide Y receptor type 2 (Y2R) agonist, a melanocortin 4 receptor (MC4R) agonist, a 5-hydroxytryptamine receptor 2C (5-HT2CR) agonist, a G protein-coupled receptor 40 (GPR40) agonist, an antagonist or inverse agonist of the Ghrelin receptor or Cannabinoid receptor-1 (CB1R), an allosteric modulator of 5-hydroxytryptamine receptor-2C (5-HT2CR), a dual amylin and calcitonin agonists, a sodium-glucose cotransporter-2 (SGLT-2) inhibitor, polymer-bound analogs thereof, pegylated analogs thereof, and combinations thereof.

Compositions suitable for use with the present invention comprise a therapeutically effective amount of SANA, a therapeutically active amount of a GLP-1 agonist or analog, or a combination thereof, optionally in combination with a therapeutically effective amount of a third active pharmaceutical ingredient as discussed herein. The compositions can be in the form of a liquid, a solid, a powder, a vapor, or a gel that provides contact with the agents' sites of action. Suitable compositions include those capable of being administered by the oral, intravenous, subcutaneous, intramuscular, intrasternal, buccal, sublingual, transmucosal, buccal, sublingual, or rectal routes, or by inhalation. In some embodiments, the compositions are in the form of a solution, a suspension, a gel, an oil, a compressed tablet, an orally disintegrating tablet, a enteric coated tablet, a capsule, a pellet, a suppository, a powder, a lyophilized powder, an aerosol, an ointment, a cream, or a patch.

As used herein, in conjunction with one or more pharmaceutically acceptable excipients in a dosage range of 0.001 mg/kg to 1000 mg/kg of mammal (e.g. human) body weight per day in a single dose or in divided doses. One dosage range is 0.01 to 500 mg/kg body weight per day orally in a single dose or in divided doses. Administration can be delivered as individual therapeutic agents or in a combination of therapeutic agents. They can be administered alone, but typically are administered with a pharmaceutically acceptable excipient selected on the basis of the chosen route of administration and standard pharmaceutical practice.

The compositions of the present invention comprise a therapeutically effective amount of SANA, a therapeutically effective amount of a GLP-1 agonist or analog, or a combination thereof, optionally in combination with a therapeutically effective amount of a third active pharmaceutical ingredient in the form of a unit dosage form comprising one or more pharmaceutically acceptable excipients. As used herein, “pharmaceutically acceptable” refers to and compounds, materials, and the like that are safe for administration to treat a mammalian, including a human subject. Pharmaceutically acceptable excipients suitable for use with the present invention, include but are not limited to, fillers, carriers, bulking agents, vehicles, diluents, adjuvants, disintegrants, stabilizers, anti-oxidants, preservatives, lubricants, solubilizing agents, flavorants, colorants, and other readily known excipients in standard pharmaceutical practice.

Liquid preparations suitable for oral administration (e.g. suspensions, syrups, elixirs and other similar liquids) can employ media such as water, glycols, oils, alcohols, and the like. Solid preparations suitable for oral administration (e.g. powders, pills, capsules and tablets) can employ solid excipients such as starches, sugars, kaolin, lubricants, binders, disintegrating agents, antioxidants and the like.

Parenteral compositions typically employ sterile water as a carrier and optionally other ingredients, such as solubility aids. Injectable solutions can be prepared, for example, using a carrier comprising a saline solution, a glucose solution or a solution containing a mixture of saline and glucose. Further guidance for methods suitable for use in preparing pharmaceutical compositions is provided in Remington: The Science and Practice of Pharmacy. 23rd edition (Academic Press, 2020).

Therapeutic compounds can be administered in a dosage range of about 0.001 to 1000 mg/kg of mammal (e.g. human) body weight per day in a single dose or in divided doses.

One dosage range is about 0.01 to 500 mg/kg body weight per day in a single dose or in divided doses. The compositions can be provided in the form of tablets, capsules, or injectables containing about 1.0 to 500 mg of the active ingredient, particularly about 1, 5, 10, 15, 20, 25, 50, 75, 100, 150, 200, 250, 300, 400, 500, and 750 mg of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. In view of the factors affecting the specific dose level and frequency it is contemplated that the dose frequency can range from multiple doses daily to monthly dosages. The preferred dose frequency ranges from twice a day to every two weeks. A more preferred dose frequency ranges from twice a day to weekly. A most preferred dose frequency ranges from twice a day to twice a week.

In some embodiments, the present invention is directed to compositions suitable for oral administration to a mammal, and preferably a human subject, comprising a therapeutically effective amount of SANA (e.g., compound (I)) or a pharmaceutically acceptable salt thereof. In some embodiments, a therapeutically effective amount of SANA or a pharmaceutically acceptable salt thereof is 0.01 mg/day to 4,000 mg/day, 1 mg/day/4,000 mg/day, 1 mg/day to 2,000 mg/day, in particular 25 mg/day to 1,000 mg/day, particularly from 50 mg/day to 400 mg/day, and even more particularly from 80 mg/day to 300 mg/day, for example 200 mg/day. The compositions are suitable for administering SANA or a pharmaceutically acceptable salt thereof once-, twice-, thrice-daily, or four times a day, or as-directed by a health care provider. As such, in some embodiments the compositions of the present invention comprise SANA or a pharmaceutically acceptable salt thereof in an amount of about 1 mg to about 4,000 mg, about 2 mg to about 2,000 mg, about 10 mg to about 1,000 mg, about 25 mg to about 1,000 mg, about 50 mg to about 800 mg, about 75 mg to about 750 mg, about 80 mg to about 500 mg, about 100 mg to about 400 mg, about 100 mg, about 200 mg, about 250 mg, about 300 mg, about 400 mg, or about 500 mg.

In some embodiments, the compositions further comprise a therapeutically effective amount of a GLP-1 agonist or analog. The therapeutically effective amount of the GLP-1 agonist or analog can vary depending upon the specific GLP-1 agonist or analog that is administered. Generally, a therapeutically effective amount or a therapeutically effective first dose of a GLP-1 agonist or analog is a dose that has been approved by the U.S. Food & Drug Administration (“FDA”) and/or European Medicines Agency (“EMA”) as safe and effective, which can be found at www.drugs@fda.gov and www.ema.europa.eu.

As such a therapeutically effective amount of the GLP-1 agonist or analog administered, for example, once daily or once weekly, can be adapted by a person of ordinary skill in the art. For example, an oral dosage form of semaglutide was recently approved by FDA and EMA under the tradename Rybelsus®. A person of ordinary skill in the art can thus determine a therapeutically effective amount for once daily administration semaglutide by reference to the Rybelsus® prescribing information, as indicated for the improvement of glycemic control in adults with type 2 diabetes mellitus. Briefly, administration of oral semaglutide can start with 3 mg once daily for 30 days; after 30 days, the dose may be increased to 7 mg once daily; and the dose may be increased to 14 mg once daily if additional effect, such as additional glycemic control in the context of an administration for the improvement of glycemic control, is needed after at least 30 days on the 7 mg dose. Further example doses of GLP-1 agonists, for type 2 diabetes mellitus via subcutaneous injection, include: dulaglutide (Trulicity®) starting dose: 0.75 mg once weekly, maintenance dose: 0.75-4.5 mg once weekly, maximum dose: 4.5 mg once weekly; Exenatide (Byetta®) starting dose: 5 mg twice a day, maintenance dose: 5-10 mg twice a day, maximum dose: 10 mg twice daily; Exenatide (Bydureon®, Bydureon, BCise®) starting dose: 2 mg once weekly, maximum dose: 2 mg once weekly; Liraglutide (Victoza®) starting dose: 0.6 mg once daily for 1 week, maintenance dose: 1.2-1.8 mg once daily, maximum dose: 1.8 mg once daily; Semaglutide (Ozempic®) starting dose: 0.25 mg once weekly for 4 weeks. After 4 weeks of 0.25 mg, increase dose to 0.5 mg once weekly, maintenance dose: 0.5-2 mg once weekly, maximum dose: 2 mg once weekly.

In some embodiments, the compositions comprise semaglutide, liraglutide, or a combination thereof and are suitable for intravenous or subcutaneous injection. In some embodiments, a therapeutically effective dose of semaglutide or liraglutide by an intravenous or subcutaneous route is 0.25 mg/week to 2.5 mg/week with concomitant administration of compound (I) or a pharmaceutically acceptable salt thereof. In some embodiments, a therapeutically effective dose of semaglutide or liraglutide by the oral route is 0.01 mg/day to 500 mg/day, 0.1 mg/day to 250 mg/day, in particular 0.25 mg/day to 100 mg/day, particularly 0.25 mg/day to 50 mg/day, and even more particularly 3 mg/day to 15 mg/day.

In a preferred embodiment, the present invention is directed to a method of administering 200 mg/day to 400 mg/day of compound (I) or a pharmaceutically acceptable salt thereof to a subject in need thereof once- or twice-daily with concomitant daily to weekly administration of a GLP-1 agonist or analog. The present invention is also directed to unit dosage forms suitable for practicing such methods comprising compound (I) or a pharmaceutically acceptable salt thereof in an amount of 100 mg, 150 mg, or 200 mg.

In a preferred embodiment, the present invention is directed to a method of treating a subject in need thereof comprising administering 200 mg/day to 400 mg/day of compound (I) or a pharmaceutically acceptable salt thereof and administering by once-weekly injection a GLP-1 agonist or analog comprising semaglutide in a therapeutically effective amount of 0.25 mg/week to 2.4 mg/week, 0.25 mg/week, 0.5 mg/week, 1.0 mg/week, 2.0 mg/week, or 2.4 mg/week.

In a preferred embodiment, the present invention is directed to a method of treating a subject in need thereof comprising administering 200 mg/day to 300 mg/day of compound (I) or a pharmaceutically acceptable salt thereof and administering by once-weekly injection a GLP-1 agonist or analog comprising semaglutide in a therapeutically effective amount of 0.25 mg/week to 2.4 mg/week, 0.25 mg/week, 0.5 mg/week, 1.0 mg/week, 2.0 mg/week, or 2.4 mg/week.

In a preferred embodiment, the present invention is directed to a method of treating a subject in need thereof comprising administering 200 mg/day of compound (I) or a pharmaceutically acceptable salt thereof and administering by once-weekly injection a GLP-1 agonist or analog comprising semaglutide in a therapeutically effective amount of 0.25 mg/week to 2.4 mg/week, 0.25 mg/week, 0.5 mg/week, 1.0 mg/week, 2.0 mg/week, or 2.4 mg/week.

In a preferred embodiment, the present invention is directed to a method of treating a subject in need thereof comprising orally administering 50 mg/day to 300 mg/day of compound (I) or a pharmaceutically acceptable salt thereof and orally administering a GLP-1 agonist or analog comprising semaglutide in a therapeutically effective amount of 0.5 mg/day to 14 mg/day, 1 mg/day to 14 mg/day, 3 mg/day to 14 mg/day, 0.5 mg/day, 1 mg/day, 3 mg/day, 7 mg/day, or 14 mg/day.

In a preferred embodiment, the present invention is directed to a method of treating a subject in need thereof comprising orally administering 80 mg/day to 200 mg/day of compound (I) or a pharmaceutically acceptable salt thereof and orally administering a GLP-1 agonist or analog comprising semaglutide in a therapeutically effective amount of 0.5 mg/day to 14 mg/day, 1 mg/day to 14 mg/day, 3 mg/day to 14 mg/day, 0.5 mg/day, 1 mg/day, 3 mg/day, 7 mg/day, or 14 mg/day.

For example, dosage may be as follows:—compound (I) is in solid dosage form for oral administration at a dose comprised between 80 to 200 mg/day; and—the GLP-1 agonist or analog is a solid dosage form of semaglutide for oral administration at a dose at a dose of 0.5 mg/day, 1 mg/day, 3 mg/day, 7 mg/day or 14 mg/day.

In another embodiment, the present invention is directed to a method of treating a subject in need thereof comprising orally administering 50 mg/day to 400 mg/day of compound (I) or a pharmaceutically acceptable salt thereof and administering by injection a GLP-1 agonist or analog comprising dulaglutide in a therapeutically effective amount of 0.75 mg/week to 4.5 mg/week.

In another embodiment, the present invention is directed to a method of treating a subject in need thereof comprising orally administering 50 mg/day to 400 mg/day of compound (I) or a pharmaceutically acceptable salt thereof and administering by injection once daily, twice daily, or once weekly a GLP-1 agonist or analog comprising exenatide in a therapeutically effective amount of 5 mcg/day to 10 mcg/day or 2 mg/week.

In another embodiment, the present invention is directed to a method of treating a subject in need thereof comprising orally administering 50 mg/day to 400 mg/day of compound (I) or a pharmaceutically acceptable salt thereof and administering by injection once daily a GLP-1 agonist or analog comprising liraglutide in a therapeutically effective amount of 0.6 mg/day to 1.8 mg/day, 1.2 mg/day to 1.8 mg/day, 0.6 mg/day, 1.2 mg/day, or 1.8 mg/day.

In another embodiment, the present invention is directed to a method of treating a subject in need thereof comprising orally administering 50 mg/day to 400 mg/day of compound (I) or a pharmaceutically acceptable salt thereof and administering by injection once daily a GLP-1 agonist or analog comprising insulin glargine and lixisenatide in a therapeutically effective amount of 30 units/day to 60 units/day insulin glargine and about 10 mcg/day to about 66 mcg/day lixisenatide.

In another embodiment, the present invention is directed to a method of treating a subject in need thereof comprising orally administering 50 mg/day to 400 mg/day of compound (I) or a pharmaceutically acceptable salt thereof and administering by injection once daily a GLP-1 agonist or analog comprising insulin degludec and liraglutide in a therapeutically effective amount of 10 units/day to 16 units/day insulin degludec and about 0.36 mg/day to about 1.8 mg/day liraglutide.

In some embodiments, the present invention is directed to a composition suitable for oral administration to a subject in need thereof comprising 80 mg to 200 mg of compound (I) or a pharmaceutically acceptable salt thereof and a GLP-1 agonist or analog comprising semaglutide in an amount of 0.5 mg, 1 mg, 3 mg, 7 mg or 14 mg. The present invention is also directed to methods of using such compositions to treat a subject in need thereof comprising orally administering the compositions once daily to the subject. In a particularly preferred embodiment, the subject is a human.

In some embodiments, the present invention is directed to a composition suitable for oral administration to a subject in need thereof comprising 80 mg of compound (I) or a pharmaceutically acceptable salt thereof and a GLP-1 agonist or analog comprising semaglutide in an amount of 3 mg, 7 mg or 14 mg. The present invention is also directed to methods of using such compositions to treat a subject in need thereof comprising orally administering the compositions once daily to the subject. In a particularly preferred embodiment, the subject is a human.

In some embodiments, the present invention is directed to a composition suitable for oral administration to a subject in need thereof comprising 120 mg of compound (I) or a pharmaceutically acceptable salt thereof and a GLP-1 agonist or analog comprising semaglutide in an amount of 3 mg, 7 mg or 14 mg. The present invention is also directed to methods of using such compositions to treat a subject in need thereof comprising orally administering the compositions once daily to the subject. In a particularly preferred embodiment, the subject is a human.

In some embodiments, the present invention is directed to methods of treating a subject being administered a first therapeutically effective amount of a GLP-1 agonist or analog comprising administering a therapeutically effective amount of compound (I) or a pharmaceutically acceptable salt thereof and administering a second therapeutically effective amount of the GLP-1 agonist or analog, wherein the second therapeutically effective amount o the GLP-1 agonist or analog is less than the first therapeutically effective amount. Thus, in some embodiments, the present invention permits a subject in need thereof to experience the beneficial effects of a GLP-1 agonist or analog at a lower therapeutically effective daily or weekly dose. In other words, administration of compound (I) or a pharmaceutically acceptable salt thereof to a subject can permit the dose of a GLP-1 agonist or analog to be reduced, while permitting the patient to benefit from the desired therapeutic effects of compound (I) and the GLP-1 agonist or analog. In some embodiments, the methods further comprise reduction or suppression of an adverse effect caused by the GLP-1 agonist or analog while maintaining the desired therapeutic benefit. Accordingly, in some embodiments the present invention relates to the methods of administering compound (I) or a pharmaceutically acceptable salt thereof to a subject suffering from a side-effect associated with a GLP-1 agonist or analog, wherein concomitant administration of the compound (I) lowers the incidence and/or severity of such side effect(s).

The known side effects of GLP-1 agonists and analogs are generally gastrointestinal, and can include nausea, vomiting, and diarrhea, the prevalence of which has been between about 10% and 50% of subjects in both clinical studies of GLP-1 agonists and analogs, as well as post-approval reporting of side effects. Known side effects of GLP-1 agonists and analogs that can be minimized or reduced or eliminated by the methods of the present invention include, but are not limited to, a reduction in lean body mass, a reduction in muscle mass, hypoglycemia, nausea, loss of appetite, vomiting, diarrhea, abdominal pain, constipation, pancreatitis, intestinal obstruction, gastroparesis, and combinations thereof.

In some embodiments, the methods of the present invention comprise administering a second therapeutically effective amount of the GLP-1 agonist or analog concomitantly with a therapeutically effective amount of compound (I) or a pharmaceutically acceptable salt thereof, wherein the second therapeutically effective amount o the GLP-1 agonist or analog is at least 1.5-fold, at least 2-fold, at least 2.5-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5-fold, or at least 10-fold less than a first therapeutically effective amount of the GLP-1 agonist or analog that is administered without compound (I). The present invention is also directed to compositions described herein suitable for administering a therapeutically effective amount of compound (I) or a pharmaceutically acceptable salt thereof alone or in combination with a therapeutically effective amount of a GLP-1 agonist or analog to practice such methods.

In some embodiments, the present invention is also directed to treating a disease or condition in a subject in need thereof being administered a first therapeutically effective amount of a GLP-1 agonist or analog comprising administering a second therapeutically effective amount of the GLP-1 agonist or analog concomitantly with a therapeutically effective amount of compound (I) or a pharmaceutically acceptable salt thereof, wherein the second therapeutically effective amount of the GLP-1 agonist or analog is reduced by least 1.5-fold, at least 2-fold, at least 2.5-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5-fold, or at least 10-fold compared to the first therapeutically effective amount of a GLP-1 agonist or analog. The present invention is also directed to compositions described herein suitable for administering a therapeutically effective amount of compound (I) or a pharmaceutically acceptable salt thereof alone or in combination with a therapeutically effective amount of a GLP-1 agonist or analog to practice such methods.

The methods of reducing a therapeutically effective amount of a GLP-1 agonist or analog administered to a subject and/or methods of treating a subject suffering from a condition that benefits from treatment with a GLP-1 agonist or analog, in either case comprising administering a therapeutically effective amount of compound (I) or a pharmaceutically acceptable salt thereof concomitant with a second, lower dose of the GLP-1 agonist or analog wherein the second dose is at least 10%, at least 15%, at least 20%, at least 25%, at least 30%, at least 35%, at least 40%, at least 45%, at least 50% or at least 60% less than the first therapeutically effective of the GLP-1 agonist or analog that is administered without compound (I).

In particular, as exemplified herein concomitant administration and/or treatment of a subject in need thereof by administering compound (I) or a pharmaceutically acceptable salt thereof and a GLP-1 agonist or analog thereof has a synergistic effect, providing benefits in weight reduction, improved basal glucose levels and glucose tolerance compared to single treatments. In some preferred embodiments, the GLP-1 agonist or analog is liraglutide or semaglutide.

In some embodiments, concomitant administration and/or treatment of a subject in need thereof by administering compound (I) or a pharmaceutically acceptable salt thereof and a GLP-1 agonist or analog thereof prevents a regain of weight, adipose tissue, and the like following a reduction in the daily or weekly dose of a GLP-1 agonist or analog.

In some embodiments, the present invention is also directed to methods of discontinuing administration of a GLP-1 agonist or analog to a subject in need thereof comprising administering a therapeutically effective amount of compound (I) or a pharmaceutically acceptable salt thereof to the subject. For example, for subjects who wish to discontinue treatment with a GLP-1 agonist or analog due to, for example, the number and/or severity of side effects, ineffectiveness, for financial reasons, or the like, or combinations thereof, such subjects can discontinue treatment with the GLP-1 agonist or analog and instead be administered a therapeutically effective amount of compound (I) or a pharmaceutically acceptable salt thereof.

In addition to the reduction in the number and/or severity of side effects of GLP-1 agonists and analogs permitted by the present methods that enable a lower therapeutically effective dose of the GLP-1 agonist or analog, the present invention provides an alternative to long term treatment or continuous treatment with GLP-1 agonists, in that compound (I) or a pharmaceutically acceptable salt may be administered simultaneously with a GLP-1 agonist or analog followed by treatment with compound (I) alone. Thus, the present invention is directed to methods of treatment comprising sequential treatment with GLP-1 agonist or analog and compound (I), wherein the GLP-1 agonist or analog is paused temporarily or discontinued, and optionally administration of the GLP-1 agonist or analog can be resumed. In some embodiments, administration of a therapeutically effective amount of a GLP-1 agonist or analog is resumed after a pause of 1 week to 1 year, 1 month to 1 year, 2 months to 6 months, 3 months, 6, months, 9 months, or 1 year concomitant with the administration of a therapeutically effective amount of compound (I). Not being bound by any particular theory, the methods of the present invention comprising discontinuing and/or pausing treatment with a GLP-1 agonist or analog can assist with the reduction and/or management of the extent and/or severity of side effects associated with GLP-1 agonist or analog.

In some embodiments, the present invention is directed to methods of treating a subject in need thereof comprising administering compound (I) or a pharmaceutically acceptable salt as the sole therapeutic agent prior to or preferably after administering a GLP-1 agonist or analog to the same subject as the sole therapeutic agent. In some preferred embodiments, the GLP-1 agonist or analog is replaced by the therapeutically effective amount of compound (I) in order to reduce the extent and/or severity of side effects and/or to facilitate further weight loss or target weight maintenance and/or to mitigate against or reduce an increase in weight gain associated with reducing the dose of a GLP-1 agonist or analog or discontinuing treatment with a GLP-1 agonist or analog. In some preferred embodiments, the GLP-1 agonist or analog that is reduced in dose or discontinued is semaglutide or liraglutide.

In some embodiments, the administration of compound (I) as a replacement for a GLP-1 agonist or analog can be preferable to a GLP-1 agonist or analog based on the elimination or reduction in the need for regular injections of a GLP-1 agonist or analog.

In some embodiments, the present invention is directed to methods of treating a subject being administered a first therapeutically effective amount of compound (I) or a pharmaceutically acceptable salt thereof comprising administering a therapeutically effective amount of a GLP-1 agonist or analog and administering a second therapeutically effective amount of compound (I) or a pharmaceutically acceptable salt thereof, wherein the second therapeutically effective amount of compound (I) is less than the first therapeutically effective amount. Thus, in some embodiments, the present invention permits a subject in need thereof to experience the beneficial effects of compound (I) or a pharmaceutically acceptable salt thereof at a lower therapeutically effective daily dose and/or with reduced dosing frequency. In other words, administration of a GLP-1 agonist or analog to a subject can permit the dose of a compound (I) or a pharmaceutically acceptable salt thereof to be reduced, while permitting the patient to benefit from the desired therapeutic effects of compound (I) and the GLP-1 agonist or analog.

Thus, whilst side effects at effective doses of compound (I) have not been observed at any significant frequency, the concomitant administration of compound (I) and a GLP-1 agonist or analog permits the reduction in the dose of compound (I), for example during the period of simultaneous combination therapy or polytherapy, for example to 150 mg/day or less, 125 mg/day or less, 100 mg/day or less, or 80 mg/day or less. In some embodiments, such methods further comprise discontinuing administration of the GLP-1 agonist or analog and increasing the dose of compound (I) or a pharmaceutically acceptable salt thereof to those described herein, for example to about 200 mg/day.

In some embodiments, the methods of the present invention comprise administering a second therapeutically effective amount of compound (I) or a pharmaceutically acceptable salt thereof concomitantly with a therapeutically effective amount of a GLP-1 agonist or analog, wherein the second therapeutically effective amount of compound (I) or a pharmaceutically acceptable salt thereof is at least 1.5-fold, at least 2-fold, at least 2.5-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5-fold, or at least 10-fold less than a first therapeutically effective amount of compound (I) or a pharmaceutically acceptable salt thereof that is administered without the GLP-1 agonist or analog.

In some embodiments, the present invention is also directed to treating a disease or condition in a subject in need thereof being administered a first therapeutically effective amount of compound (I) or a pharmaceutically acceptable salt thereof comprising administering a second therapeutically effective amount of compound (I) or a pharmaceutically acceptable salt thereof concomitantly with a therapeutically effective amount of a GLP-1 agonist or analog, wherein the second therapeutically effective amount of compound (I) or a pharmaceutically acceptable salt thereof is reduced by least 1.5-fold, at least 2-fold, at least 2.5-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5-fold, or at least 10-fold compared to the first therapeutically effective amount of compound (I) or a pharmaceutically acceptable salt thereof.

The methods of reducing a therapeutically effective amount of compound (I) or a pharmaceutically acceptable salt thereof administered to a subject and/or methods of treating a subject suffering from a condition that benefits from treatment with compound (I) or a pharmaceutically acceptable salt thereof, in either case comprising administering a therapeutically effective amount of a GLP-1 agonist or analog concomitant with a second, lower dose of compound (I) or a pharmaceutically acceptable salt thereof wherein the second dose is at least 10%, at least 15%, at least 20%, at least 25%, at least 30%, at least 35%, at least 40%, at least 45%, at least 50% or at least 60% less than the first therapeutically effective of compound (I) or a pharmaceutically acceptable salt thereof that is administered without the GLP-1 agonist or analog.

In the methods of various embodiments, pharmaceutical compositions including the active agents can be administered to a subject in an “effective amount.” An effective amount may be any amount that provides a beneficial effect to the patient, and in particular embodiments, the effective amount is an amount that may: (1) prevent the subject from experiencing weight gain; (2) reduce the rate of weight gain in a subject; (3) induce weight loss in a subject; (4) treat the subject for one or more adverse effects associated with weight gain and/or obesity, such as, but not limited to glucose intolerance; (5) treat obesity; (6) induce thermogenesis in a subject.

Pharmaceutical formulations containing the compounds of the invention and a suitable carrier can be in various forms including, but not limited to, solids, solutions, powders, fluid emulsions, fluid suspensions, semi-solids, and dry powders including an effective amount of an the active agents of the invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, antioxidants, preservatives and the like. The means and methods for administration are known in the art and an artisan can refer to various pharmacologic references for guidance. For example, Modern Pharmaceutics, Banker & Rhodes, 4th edition, CRC Press (1996); and Goodman & Gilman's, The Pharmaceutical Basis of Therapeutics, 14th Edition, McGraw Hill/Medical (2022) both of which are hereby incorporated by reference in their entireties can be consulted.

Other embodiments of the invention include the active agents prepared as described above which are formulated as a solid dosage form for oral administration including capsules, tablets, pills, powders, and granules. In such embodiments, the active compound may be admixed with one or more inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents and can additionally be prepared with enteric coatings.

In another exemplary embodiment, an oily preparation of an active agent prepared as described above may be lyophilized to form a solid that may be mixed with one or more pharmaceutically acceptable excipient, carrier or diluent to form a tablet, and in yet another embodiment, the active agents may be crystallized to from a solid which may be combined with a pharmaceutically acceptable excipient, carrier or diluent to form a tablet.

The means and methods for tableting are known in the art and one of ordinary skill in the art can refer to various references for guidance. For example, Pharmaceutical Manufacturing Handbook: Production and Processes, Shayne Cox Gad, John Wiley & Sons, Inc., Hoboken, New Jersey (2008), which is hereby incorporated by reference in its entirety can be consulted.

Further embodiments which may be useful for oral administration of the active agents include liquid dosage forms. In such embodiments, a liquid dosage may include a pharmaceutically acceptable emulsion, solution, suspension, syrup, and elixir containing inert diluents commonly used in the art, such as water. Such compositions may also comprise adjuvants, such as wetting agents, emulsifying and suspending agents, and sweetening, flavoring, and perfuming agents. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt. Other suitable diluents include, but are not limited to those described below:

Vegetable oil: As used herein, the term “vegetable oil” refers to a compound, or mixture of compounds, formed from ethoxylation of vegetable oil, wherein at least one chain of polyethylene glycol is covalently bound to the vegetable oil. In some embodiments, the fatty acids may have between about twelve carbons to about eighteen carbons. In some embodiments, the amount of ethoxylation can vary from about 2 to about 200, about 5 to 100, about 10 to about 80, about 20 to about 60, or about 12 to about 18 of ethylene glycol repeat units. The vegetable oil may be hydrogenated or unhydrogenated. Suitable vegetable oils include, but are not limited to castor oil, hydrogenated castor oil, sesame oil, corn oil, peanut oil, olive oil, sunflower oil, safflower oil, soybean oil, benzyl benzoate, sesame oil, cottonseed oil, and palm oil. Other suitable vegetable oils include commercially available synthetic oils such as, but not limited to, Miglyol™ 810 and 812 (available from Dynamit Nobel Chemicals, Sweden) Neobee™ MS (available from Drew Chemical Corp.), Alofine™ (available from Jarchem Industries), the Lubritab™ series (available from JRS Pharma), the Sterotex™ (available from Abitec Corp.), Softisan™ 154 (available from Sasol), Croduret™ (available from Croda), Fancol™ (available from the Fanning Corp.), Cutina™ HR (available from Cognis), Simulsol™ (available from CJ Petrow), EmCon™ CO (available from Amisol Co.), Lipvol™ CO, SES, and HS-K (available from Lipo), and Sterotex™ HM (available from Abitec Corp.). Other suitable vegetable oils, including sesame, castor, corn, and cottonseed oils, include those listed in P. J. Sheskey, B. C. Hancock, G. P. Moss, D. J. Goldfarb, Handbook of Pharmaceutical Excipients, (2020), 9th ed., which is incorporated herein by reference in its entirety. Suitable polyethoxylated vegetable oils, include but are not limited to, Cremaphor™ EL or RH series (available from BASF), Emulphor™ EL-719 (available from Stepan products), and Emulphor™ EL-620P (available from GAF).

Mineral oils: As used herein, the term “mineral oil” refers to both unrefined and refined (light) mineral oil. Suitable mineral oils include, but are not limited to, the Avatech™ grades (available from Avatar Corp.), Drakeol™ grades (available from Penreco), Sirius™ grades (available from Shell), and the Citation™ grades (available from Avater Corp.).

Castor oils: As used herein, the term “castor oil,” refers to a compound formed from the ethoxylation of castor oil, wherein at least one chain of polyethylene glycol is covalently bound to the castor oil. The castor oil may be hydrogenated or unhydrogenated. Synonyms for polyethoxylated castor oil include, but are not limited to polyoxyl castor oil, hydrogenated polyoxyl castor oil, mcrogolglyceroli ricinoleas, macrogolglyceroli hydroxystearas, polyoxyl 35 castor oil, and polyoxyl 40 hydrogenated castor oil. Suitable polyethoxylated castor oils include, but are not limited to, the Nikkol™ HCO series (available from Nikko Chemicals Co. Ltd.), such as Nikkol HCO-30, HC-40, HC-50, and HC-60 (polyethylene glycol-30 hydrogenated castor oil, polyethylene glycol-40 hydrogenated castor oil, polyethylene glycol-SO hydrogenated castor oil, and polyethylene glycol-60 hydrogenated castor oil, Emulphor™ EL-719 (castor oil 40 mole-ethoxy late, available from Stepan Products), the Cremophore™ series (available from BASF), which includes

Cremophore REMO, RH60, and EL35 (polyethylene glycol-40 hydrogenated castor oil, polyethylene glycol-60 hydrogenated castor oil, and polyethylene glycol-35 hydrogenated castor oil, respectively), and the Emulgin® RO and HRE series (available from Cognis PharmaLine). Other suitable polyoxyethylene castor oil derivatives include those listed in R. C. Rowe and P. J. Shesky, Handbook of Pharmaceutical Excipients, (2006), 5th ed., which is incorporated herein by reference in its entirety.

Sterol: As used herein, the term “sterol” refers to a compound, or mixture of compounds, derived from the ethoxylation of sterol molecule. Suitable polyethoyxlated sterols include, but are not limited to, PEG-24 cholesterol ether, Solulan™ C-24 (available from Amerchol); PEG-30 cholestanol, Nikkol™DHC (available from Nikko); Phytosterol, GENEROL™ series (available from Henkel); PEG-25 phyto sterol, Nikkol™ BPSH-25 (available from Nikko); PEG-5 soya sterol, Nikkol™ BPS-5 (available from Nikko); PEG-JO soya sterol, Nikkol™ BPS-JO (available from Nikko); PEG-20 soya sterol, Nikkol™ BPS-20 (available from Nikko); and PEG-30 soya sterol, Nikkol™ BPS-30 (available from Nikko).

Polyethylene glycol: As used herein, the term “polyethylene glycol” or “PEG” refers to a polymer containing ethylene glycol monomer units of formula -0-CH2-CH2-. Suitable polyethylene glycols may have a free hydroxyl group at each end of the polymer molecule, or may have one or more hydroxyl groups etherified with a lower alkyl, e.g., a methyl group. Also suitable are derivatives of polyethylene glycols having esterifiable carboxy groups. Polyethylene glycols useful in the present invention can be polymers of any chain length or molecular weight, and can include branching. In some embodiments, the average molecular weight of the polyethylene glycol is from about 200 to about 9000. In some embodiments, the average molecular weight of the polyethylene glycol is from about 200 to about 5000. In some embodiments, the average molecular weight of the polyethylene glycol is from about 200 to about 900. In some embodiments, the average molecular weight of the polyethylene glycol is about 400. Suitable polyethylene glycols include, but are not limited to polyethylene glycol-200, polyethylene glycol-300, polyethylene glycol-400, polyethylene glycol-600, and polyethylene glycol-900. The number following the dash in the name refers to the average molecular weight of the polymer. In some embodiments, the polyethylene glycol is polyethylene glycol-400. Suitable polyethylene glycols include, but are not limited to the Carbowax™ and Carbowax™ Sentry series (available from Dow), the Lipoxol™ series (available from Brenntag), the Lutrol™ series (available from BASF), and the Pluriol™ series (available from BASF).

Propylene glycol fatty acid ester: As used herein, the term “propylene glycol fatty acid ester” refers to a monoether or diester, or mixtures thereof, formed between propylene glycol or polypropylene glycol and a fatty acid. Fatty acids that are useful for deriving propylene glycol fatty alcohol ethers include, but are not limited to, those defined herein. In some embodiments, the monoester or diester is derived from propylene glycol. In some embodiments, the monoester or diester has about 1 to about 200 oxypropylene units. In some embodiments, the polypropylene glycol portion of the molecule has about 2 to about 100 oxypropylene units. In some embodiments, the monoester or diester has about 4 to about 50 oxypropylene units. In some embodiments, the monoester or diester has about 4 to about 30 oxypropylene units. Suitable propylene glycol fatty acid esters include, but are not limited to, propylene glycol laurates: Lauroglycol™ FCC and 90 (available from Gattefosse); propylene glycol caprylates: Capryol™ PGMC and 90 (available from Gatefosse); and propylene glycol dicaprylocaprates: Labrafac™ PG (available from Gatefosse).

Stearoyl macrogol glyceride: Stearoyl macrogol glyceride refers to a polyglycolized glyceride synthesized predominately from stearic acid or from compounds derived predominately from stearic acid, although other fatty acids or compounds derived from other fatty acids may be used in the synthesis as well. Suitable stearoyl macrogol glycerides include, but are not limited to, Gelucire® 50/13 (available from Gattefosse).

In some embodiments, a diluent for use with the composition of the present invention comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, a metal aluminosilicate, and combinations thereof.

Suitable pharmaceutically acceptable excipients and/or carriers for use in solid and/or liquid dosage forms of the present invention include, but are not limited to: sorbitol (including by not limited to PharmSorbidex E420 (available from Cargill), Liponic 70-NC and 76-NC (available from Lipo Chemical), Neosorb (available from Roquette), Partech SI (available from Merck), and Sorbogem (available from SPI Polyols)), starch, sodium starch glycolate, and pregelatinized starch include, but are not limited to, those described in R. C. Rowe and P. J. Shesky, Handbook of Pharmaceutical Excipients, 5 (2006), 5th ed., which is incorporated herein by reference in its entirety.

Suitable pharmaceutically acceptable disintegrants for use in dosage forms of the present invention include, but are not limited to, croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floe, carboxymethylcellulose, hydroxypropylcellulose, calcium silicate, a metal carbonate, sodium bicarbonate, calcium citrate, calcium phosphate, and combinations thereof.

Still further embodiments of the invention include the combination products of the invention administered in combination with other active such as, for example, adjuvants, protease inhibitors, or other compatible drugs or compounds where such combination is seen to be desirable or advantageous in achieving the desired effects of the methods described herein.

Other embodiments of the present invention include a pharmaceutical composition comprising effective amounts of the active agents and one or more pharmaceutically acceptable excipient. Other embodiments include a pharmaceutical composition comprising an effective amount of pharmaceutically-acceptable salts of the active agent. Other embodiments include a pharmaceutical composition comprising an effective amount of pharmaceutically-acceptable salts of active agents and a pharmaceutically-acceptable excipient.

In yet other embodiments, the combination products of the invention may be combined with one or more further therapeutic agents. Further therapeutic agents my include but are not limited to: an anti-platelet agent, an inhibitor of angiotensin II, an ACE inhibitor, a Ca++channel blocker, an insulin sensitizer, a HMG-CoA reductase inhibitor, a beta blocker, a non steroidal anti-inflammatory drug, a steroidal anti-inflammatory drug, peroxisome proliferator-activated receptors (PPAR) modulators, and combinations thereof.

The combination products of the invention and pharmaceutical compositions thereof as described herein may be administered to subjects to treat obesity. In other embodiments, the combination products of the invention and pharmaceutical compositions thereof as described herein may be administered to subjects to prevent weight gain or reduce the rate of weight gain in a subject. In some embodiments, the combination products of the invention and pharmaceutical compositions thereof as described herein may be administered to induce weight loss in a subject. In some embodiments, the combination products of the invention and pharmaceutical compositions thereof as described herein may be administered to treat the subject for one or more adverse effects associated with weight gain and/or obesity, such as, but not limited to glucose intolerance. In some embodiments, the combination products of the invention and pharmaceutical compositions thereof as described herein may be administered to treat obesity. In some embodiments, the combination products of the invention and pharmaceutical compositions thereof as described herein may be administered to induce thermogenesis in a subject.

The following examples disclosed detailed methods of practicing the methods of the present invention, as well as exemplary compositions for practicing such methods. In particular, the methods described herein are used in order to demonstrate biological activity and therapeutic use and are presented for illustrative purposes only, without any intention to restrict or otherwise limit the scope of the invention. All parts are by weight and temperatures are in degrees Celsius unless otherwise indicated.

### EXAMPLES

Methods of preparing compound (I) or pharmaceutically acceptable salts thereof are described in, for example, U.S. Pat. Nos. 10,537,537 and 11,400,066, the disclosure of which is incorporated herein by reference in the entirety.

All studies conducted in mice utilized C57BL/6J mice maintained at the Institut Pasteur de Montevideo Animal facility (UBAL). Experimental protocols were approved by the Institutional Animal Care and Use Committee of the Institut Pasteur de Montevideo (CEUA, Protocol numbers 003-19 and 006-19). Studies were performed according to the methods approved in the protocols. All the experiments were performed on adult male mice (10-12 weeks of age) and were conducted at either 22° C. with free access to food and water. Unless specified, mice were housed at 22° C. in groups of five.

High-fat diet (HFD) feeding: Male C57BL/6J mice housed at 22° C. were fed a high-fat diet (42% fat and 0.25% cholesterol, AlN93G, LabDiet, USA), starting at 12 weeks of age and for a duration of 5 weeks prior to beginning administration of any active pharmaceutical ingredients. After the onset of the treatment, HFD ad libitum was maintained until the end of the experiments.

Drug delivery: SANA was administered orally (PO) by mixing with the food (50, 100, or 200 mg/kg/day), or subcutaneously (SC, 10, 15, or 20 mg/kg/day in 100 mM phosphate buffer, pH 6.5). In all cases the vehicle was used as control. Liraglutide (Novo Nordisk, Denmark) was administered daily, subcutaneously (SC, 0.2 mg/kg/day). Semaglutide (Ozempic®, Novo Nordisk, Denmark) was administered subcutaneously (SC, 2 nmol/kg/day or 40 mg/kg every 3 days).

Blood glucose measurements: Mice were kept in fasting for 16 h before fasting glucose measurement and glucose tolerance tests (GTT). When performing GTT, mice were injected (IP) with 1.5 g/kg body weight of glucose solution. Plasma glucose concentrations were measured from blood obtained from the tail using a hand-held glucometer (Accu-Chek, Roche).

### Example 1: In Vivo Synergistic Action of SANA and Liraglutide

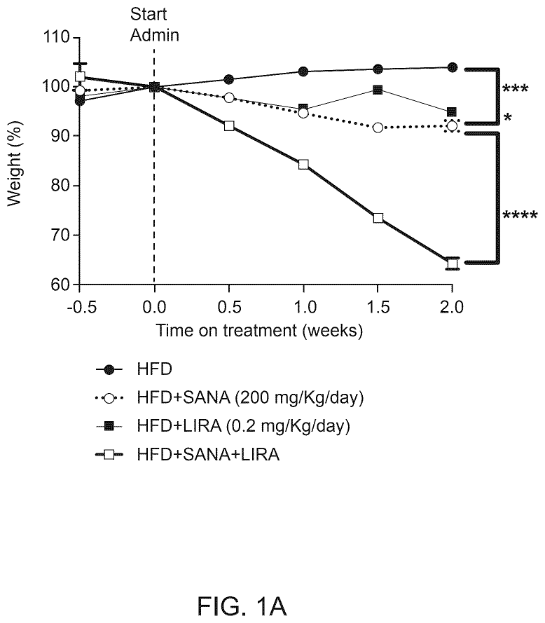

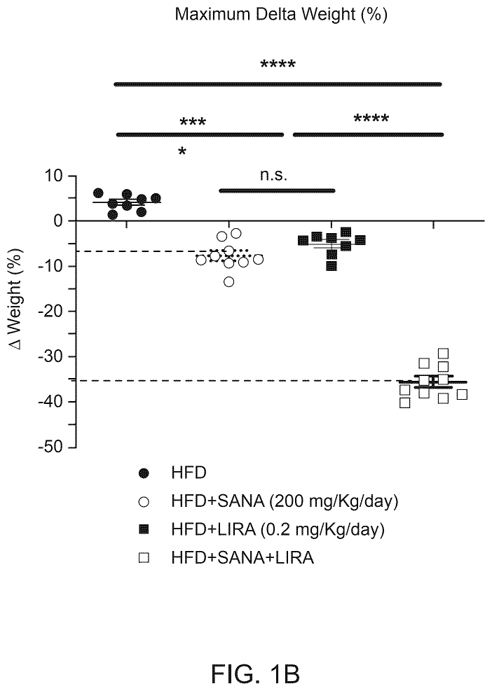

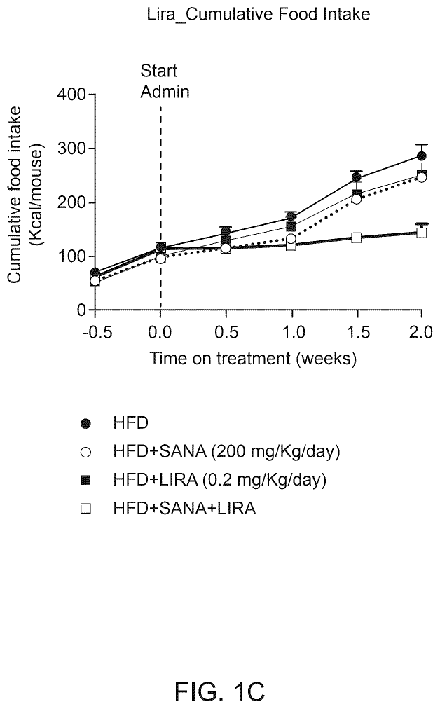

Mice were fed with a high fat diet (HFD) for 5 weeks prior to the initiation of treatment, and the high-fat diet continued during a 2-week treatment period. FIGS. 1A-1B show graphs of average mouse weight without treatment (HFD, n=8), treatment with SANA (HFD+SANA, 200 mg/kg/day, n=9), treatment with liraglutide (HFD+LIRA, 0.2 mg/kg/day, n=8), or treatment with a combination of SANA (200 mg/kg/day) and liraglutide (0.2 mg/kg/day) (HFD+SANA+LIRA, n=10).

Referring to FIGS. 1A and 1B, obese mice administered SANA alone (“HFD+SANA”) or liraglutide alone (“HFD+LIRA”) experienced a weight loss of about 10% after two weeks. In contrast, obese mice administered concomitant SANA and liraglutide (“HFD+SANA+LIRA”) experienced an average weight loss of about 35%, demonstrating the synergistic effects of the methods of the present invention.

As shown in FIG. 1C, cumulative food intake increased significantly for the obese mice on a high fat diet, as well as those receiving SANA alone (“HFD+SANA”) or liraglutide alone (“HFD+LIRA”), increasing by a factor of roughly 2-2.5. In contrast, obese mice administered concomitant SANA and liraglutide (“HFD+SANA+LIRA”) demonstrated a cumulative food intake increasing by a factor of only 1.3-1.4, further the synergistic effects of the methods of the present invention.

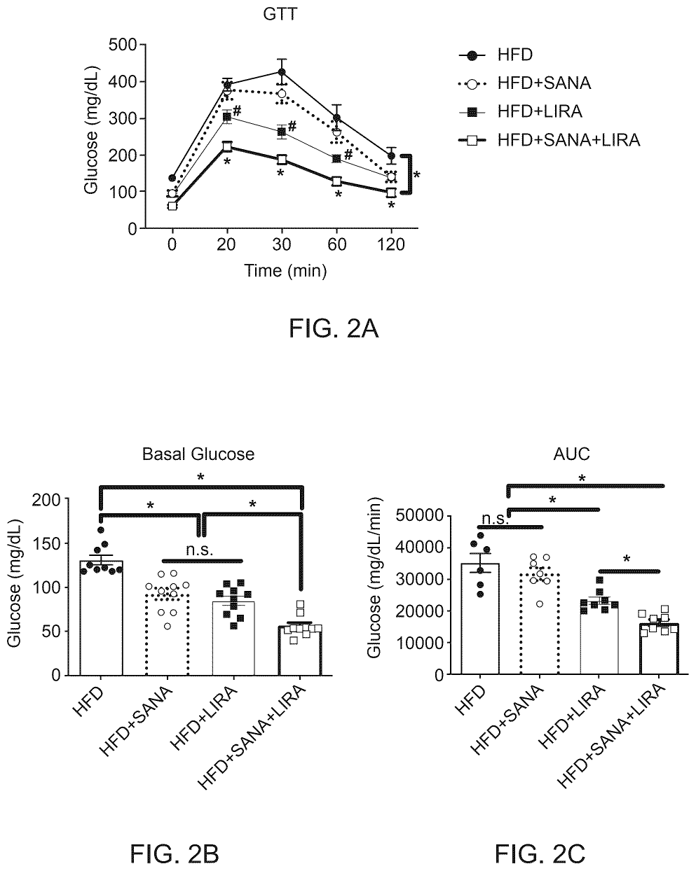

Glucose tolerance (“GTT”), basal glucose, and glucose Area Under the Curve (“AUC”) were also measured following two weeks of treatment, and the results are presented graphically in FIGS. 2A, 2B, and 2C with * indicating a statistically significant difference, p<0.05.

Referring to FIGS. 2A and 2B, concomitant administration of SANA (200 mg/kg/day) and liraglutide (0.2 mg/kg/day) improved glucose tolerance and basal glucose levels, respectively, in obese mice compared to either agent administered alone. Referring to FIG. 2C, glucose AUC was also reduced in obese mice by concomitant administration of SANA and liraglutide compared to no treatment or either active agent administered alone.

### Example 2A: In Vivo Synergistic Action of SANA and Semaglutide

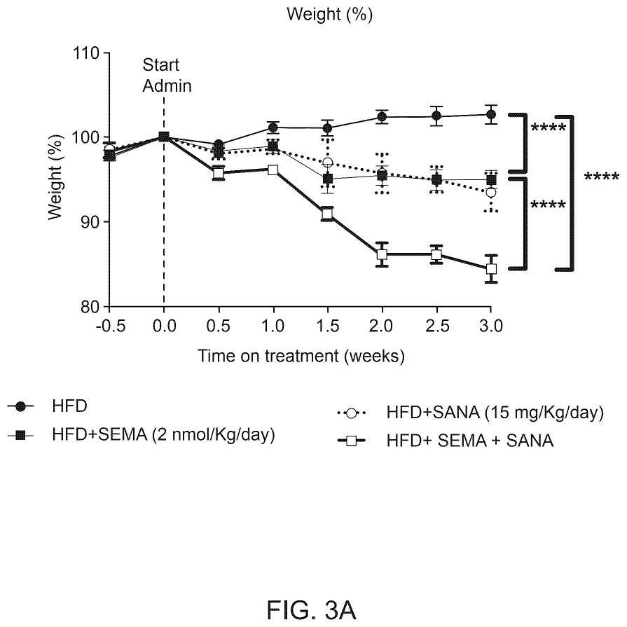

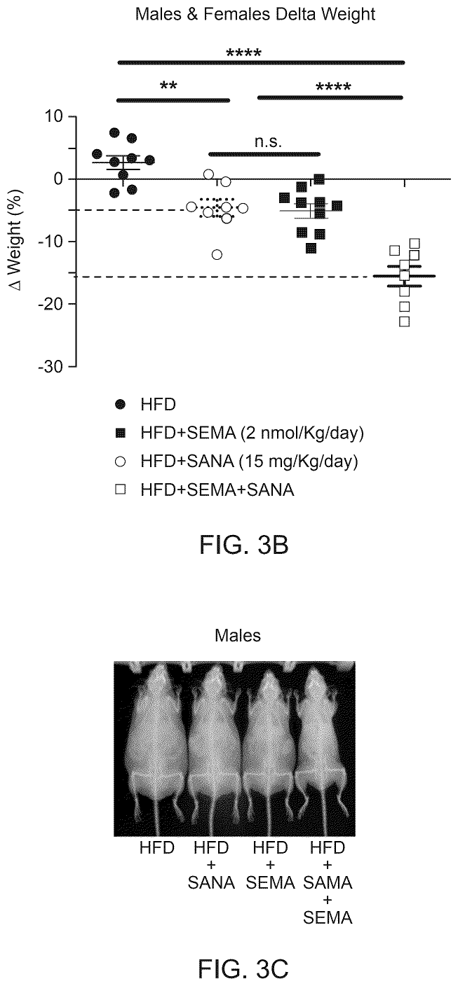

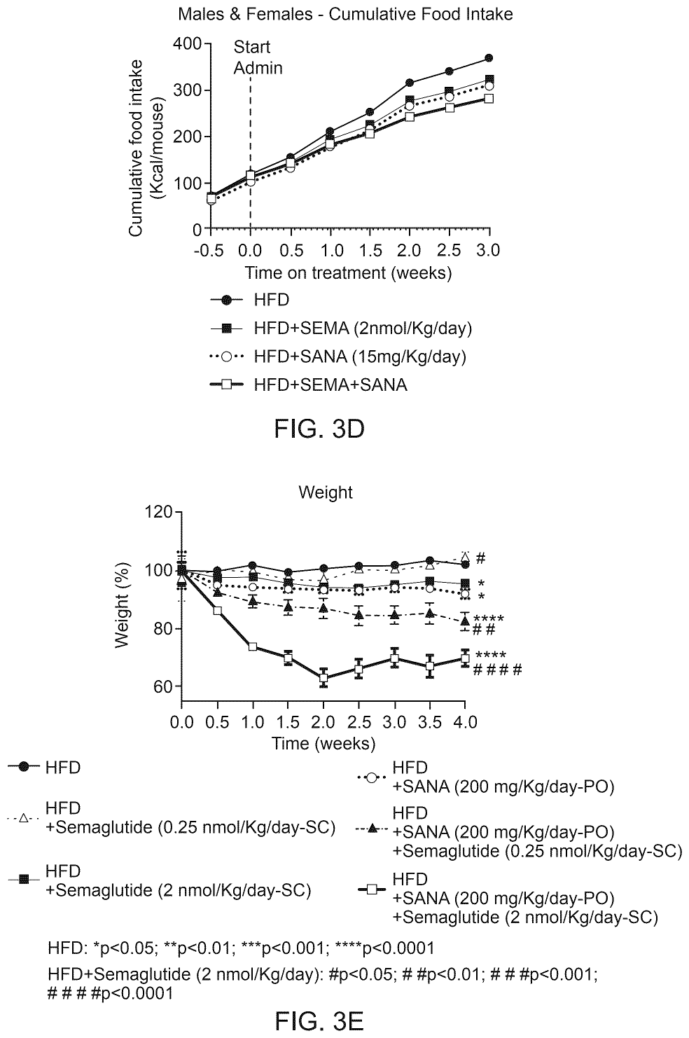

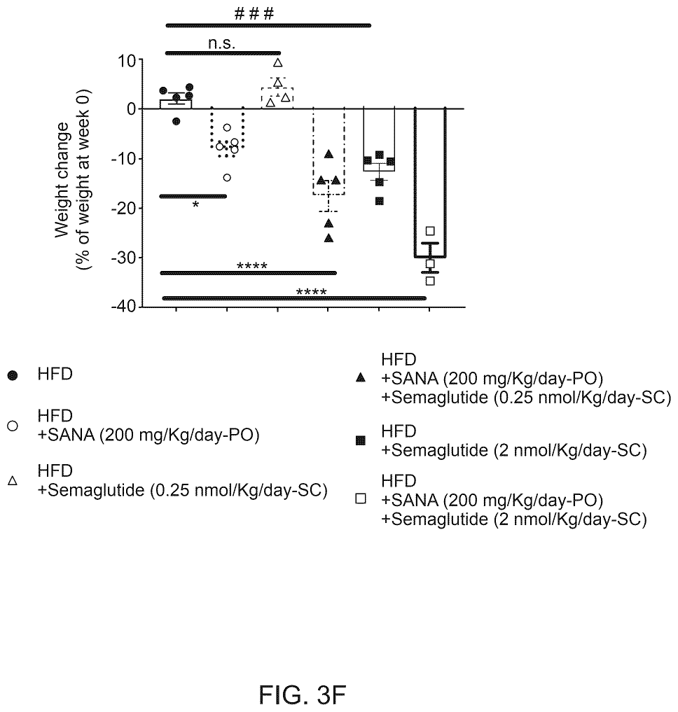

Similar effects were seen with concomitant administration of SANA and semaglutide. FIGS. 3A and 3B show graphs of the average mouse weight without treatment (n=9), treatment with SANA (15 mg/kg/day, n=8), treatment with semaglutide (2 nmol/kg/day, n=10), or treatment with a combination of SANA (15 mg/kg/day) and semaglutide (2 nmol/kg/day) (n=10). Mice were fed with a high fat diet (HFD) for 5.5 weeks prior to the initiation of treatment, and the high-fat diet continued during the 3-week treatment period. Referring to FIGS. 3A and 3B, obese mice administered SANA alone (HFD+SANA) or semaglutide alone (HFD+SEMA) experienced an average weight loss of about 5% after three weeks. In contrast, obese mice administered concomitant SANA and semaglutide (HFD+SEMA+SANA) experienced an average weight loss of about 15%, as can be seen in FIG. 3C. Further, as shown in FIG. 3D, cumulative food intake was lowest for the mice receiving concomitant SANA and semaglutide, at roughly 15% lower than the untreated group. These results also demonstrate the synergistic effects of the methods of the present invention, which are similar to those observed synergistic effect as seen with liraglutide.

In a separate experiment, different doses of subcutaneous semaglutide alone were compared to treatment with oral administration of SANA alone, and to treatment with a combination of oral SANA and two different doses of subcutaneous semaglutide. Specifically, mice were fed with a high fat diet (HFD) for 5.5 weeks prior to the initiation of treatment, and the high-fat diet continued during the 4-week treatment period the average mouse weight without treatment (n=5), treatment with SANA (200 mg/kg/day, n=5), treatment with semaglutide (0.25 nmol/kg/day, n=5), treatment with semaglutide (2 nmol/kg/day, n=4), treatment with a combination of SANA (200 mg/kg/day) and semaglutide (0.25 nmol/kg/day) (n=5), or treatment with a combination of SANA (200 mg/kg/day) and semaglutide (2 nmol/kg/day) (n=3). Referring to FIGS. 3E and 3F, obese mice administered SANA alone (HFD+SANA) or 2 nmol/kg/day of semaglutide alone experienced an average weight loss of about 5% after four weeks. Obese mice administered 0.25 nmol/kg/day of semaglutide alone gained roughly 2% at four weeks. In contrast, obese mice administered concomitant SANA and 0.25 nmol/kg/day semaglutide experienced an average weight loss of about 15%, and obese mice receiving concomitant SANA and 2 nmol/kg/day experienced a weight loss of roughly 30%. These results demonstrate the synergistic effects of the methods of the present invention, which are similar to those observed synergistic effect as seen with liraglutide.

### Example 2B: In Vivo Synergistic Action of SANA and Semaglutide

Concomitant administration of SANA (s.c. every day) and semaglutide (s.c. every three days) provided similar synergistic effects. Mice were fed a high-fat diet for 5.5 weeks, and then randomized to receive SANA (10 mg/kg/day), semaglutide (40 mg/kg every 3 days), concomitant administration of SANA (10 mg/kg/day) plus semaglutide (40 mg/kg every 3 days), or no treatment (control) for a period of 1.5 weeks.

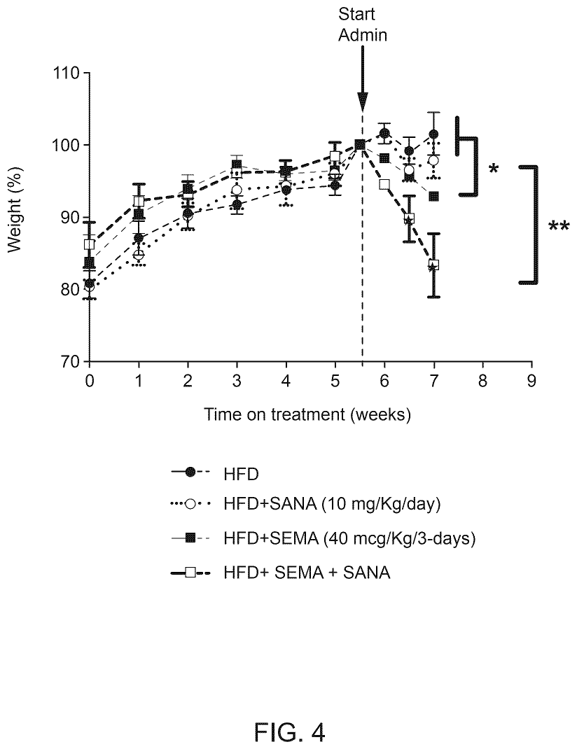

Referring to FIG. 4, the results shows that even for a short period of treatment, concomitant administration of SANA with a GLP-1 agonist (semaglutide) achieved significantly better weight loss compared to either treatment alone, which demonstrate the synergistic effects of the methods of the present invention.

### Example 3: Comparison of Weight Regain Between SANA and GLP-1 Agonist

Comparison of weight regain in mice following discontinuation of treatment with SANA or a GLP-1 agonist was measured in order to quantify the “rebound effect” as a result of discontinuing treatment. Mice were fed a high-fat diet for a period of 5 weeks, and were then untreated (HFD), treated with GLP-1 agonist (liraglutide, 0.2 mg/kg/day SC daily injection, LIRA), or treated with SANA (200 mg/kg/day PO) for a period of nine weeks. The high-fat diet was continued during the treatment period. Following week 9, all treatment was discontinued and weight regain in the mice was monitored through week 14.

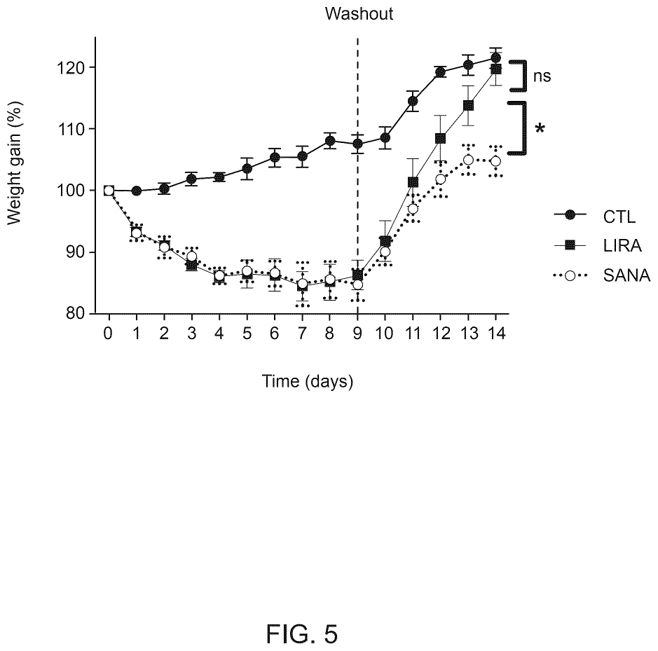

Referring to FIG. 5, the results show that mice treated with either liraglutide or SANA experienced similar weight loss during the treatment period, with an average of 15% reduction in weight. However, following discontinuation of treatment, mice that were administered liraglutide re-gained on average 15% more weight than mice treated with SANA (with * showing statistically significant difference, p<0.05).

### Example 4: SANA Administration to Control Weight Regain

The ability of SANA to prevent the regain of weight in mice following discontinuation of GLP-1 treatment was also assessed. Mice were fed a high-fat diet for a period of 5.5 weeks, and were then untreated (HFD), treated with GLP-1 agonist (semaglutide, 40 mg/kg s.c. every 3 days), or treated with semaglutide+SANA (10 mg/kg/day) for a period of 2.5 weeks. At week 8, except for the control group (HFD) all groups were treated only with SANA (10 mg/kg/day, s.c.).

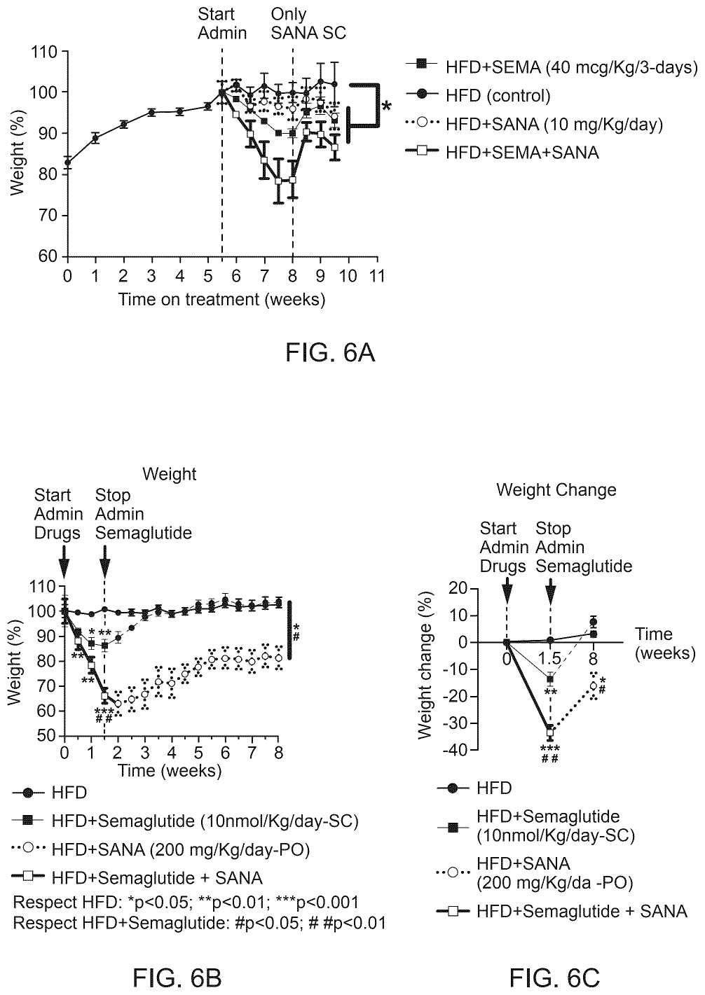

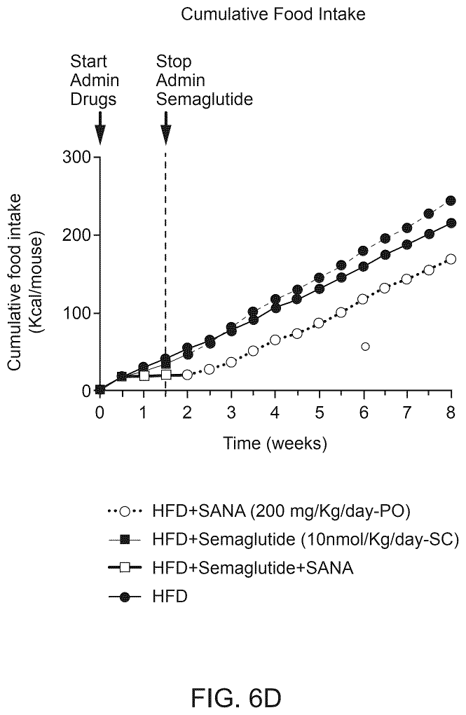

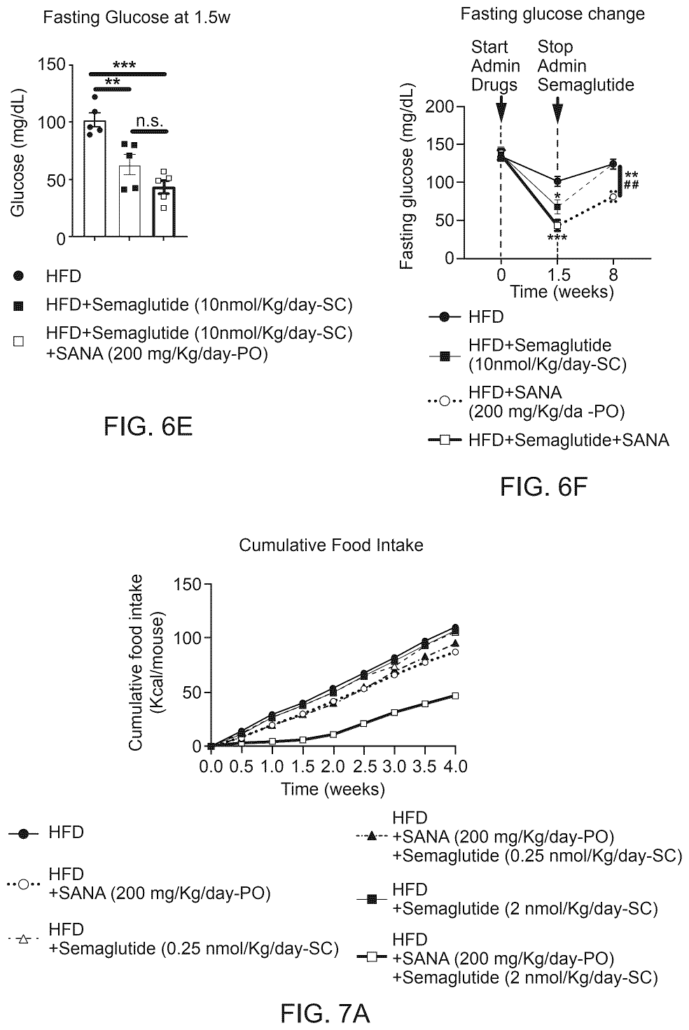

FIG. 6A shows that the administration of SANA following discontinuation of treatment with semaglutide alone or treatment with concomitant SANA plus semaglutide, prevented regain of weight. * indicates statistically significant difference, p<0.05.

A higher dose of SANA was then assessed for its ability to prevent the regain of weight in mice following discontinuation of GLP-1 treatment. Referring to FIGS. 6B and 6C, mice were fed a high-fat diet for a period of 5.5 weeks, and then were untreated (HFD), treated with GLP-1 agonist (semaglutide, 10 nmol/kg/day—SC), or treated with semaglutide+SANA for a period of 1.5 weeks, at which time, the semaglutide treatment was ceased and the groups were treated only with SANA (200 mg/kg/day, PO). The weight regain of the mice was monitored for another 1.5-6.5 weeks. *, **, ***, #, and ## show statistically significant differences, p<0.05, p<0.01, p<0.001, p<0.05, and p<0.01, respectively. These results further demonstrate that the administration of SANA following discontinuation of treatment with semaglutide alone or concomitant SANA plus semaglutide prevented regain of weight.

Further, as shown in FIG. 6D, cumulative food intake remained lower with administration of SANA following discontinuation of treatment with concomitant SANA plus semaglutide. Fasting glucose was also significantly improved with administration of SANA following discontinuation of treatment with concomitant SANA plus semaglutide in comparison to semaglutide alone, being 55% lower versus 45% lower, as shown in FIGS. 6E and 6F.

These results show that SANA is effective as a replacement therapy after semaglutide or semaglutide+SANA treatment.

### Example 5: Cumulative Food Intake, Glucose Levels, and Serum NEFA

As described in Example 2A above, mice were fed with a high fat diet (HFD) for 5.5 weeks prior to the initiation of treatment, and the high-fat diet continued during the 4 week treatment period. Different doses of semaglutide were provided, either alone or in combination with SANA, and compared to SANA alone and untreated mice alone. Specifically, mice were either untreated (HFD) (n=5), or treated with SANA (200 mg/kg/day, n=5), semaglutide (0.25 nmol/kg/day, n=5), semaglutide (2 nmol/kg/day, n=4), a combination of SANA (200 mg/kg/day) and semaglutide (0.25 nmol/kg/day) (n=5), or a combination of SANA (200 mg/kg/day) and semaglutide (2 nmol/kg/day) (n=3).

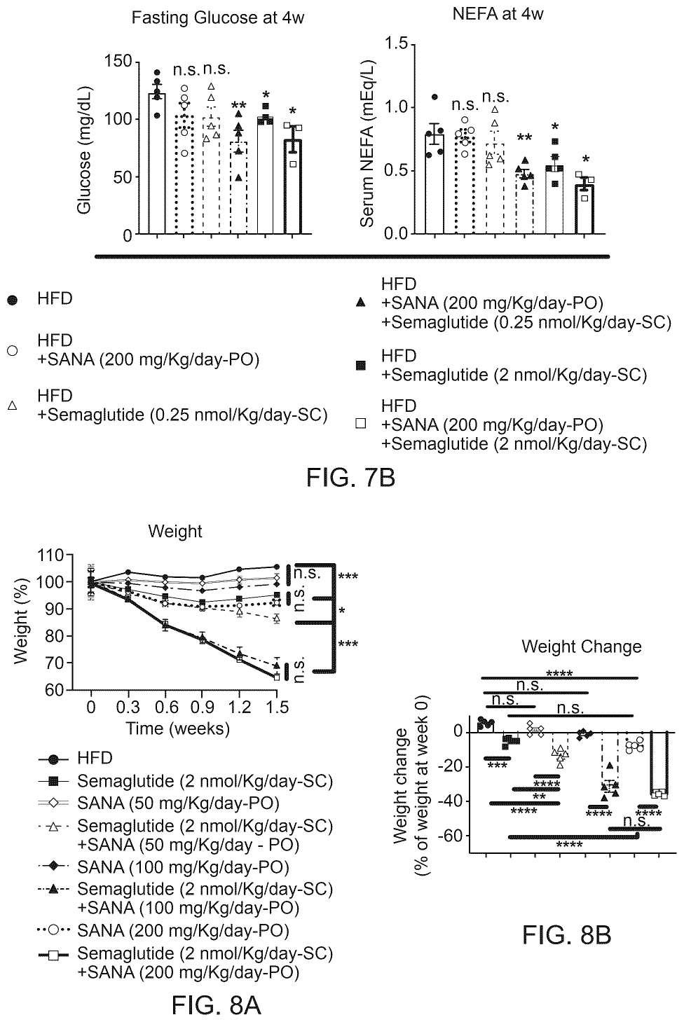

Referring to FIG. 7A, obese mice administered 2 nmol/kg/day of semaglutide alone demonstrated little to no difference in cumulative food intake at week 4 in comparison to the untreated mice. Mice treated with SANA alone or SANA and 0.25 nmol/kg/day semaglutide demonstrated a cumulative food intake roughly 5-10% less than the untreated group at week 4. However, mice treated with SANA and 2 nmol/kg/day semaglutide showed a cumulative food intake of more than 50% less than the untreated group at week 4. Again, these results demonstrate the synergistic effects of the present invention.

Further, FIG. 7B shows fasting glucose and serum non-esterified fatty acids (NEFA) at 4 weeks in the aforementioned treatment groups. In mice treated with SANA alone, 0.25 nmol/kg/day semaglutide alone, or 2 nmol/kg/day semaglutide alone, fasting glucose levels were decreased by roughly 20-25% compared to untreated mice. In contrast, mice treated with a combination of SANA and 0.25 nmol/kg/day semaglutide or SANA in combination with 2 nmol/kg/day semaglutide demonstrated fasting glucose levels 30-40% below those of untreated mice. Likewise, the mice treated with SANA alone or 0.25 nmol/kg/day semaglutide showed little to no change in NEFA levels at 4 weeks. Mice treated with 2 nmol/kg/day semaglutide showed a roughly 25-28% drop in serum NEFA levels at 4 weeks. Mice treated with a combination of SANA and 0.25 nmol/kg/day semaglutide showed approximately 40% drop in serum NEFA at 4 weeks, and those treated with SANA in combination with 2 nmol/kg/day semaglutide showed a drop of 45-50% at 4 weeks. These results further demonstrate the synergistic effects of the present invention.

### Example 6: Cumulative Food Intake, Glucose Levels, and Serum NEFA

Next, different doses of SANA were tested, either alone or in combination with semaglutide, and compared to treatment with semaglutide alone and untreated mice. Specifically, as described in Example 2A above, mice were fed with a high fat diet (HFD) for 5.5 weeks prior to the initiation of treatment, and the high-fat diet continued during the 1.5-week treatment period. Mice were either untreated (HFD) (n=5), or treated with 50 mg/kg/day SANA (n=5), 100 mg/kg/day SANA (n=5), 200 mg/kg/day SANA (n=5), semaglutide (2 nmol/kg/day, n=5), a combination of 50 mg/kg/day SANA and semaglutide (2 nmol/kg/day) (n=5), a combination of 100 mg/kg/day SANA and semaglutide (2 nmol/kg/day) (n=5), or a combination of 200 mg/kg/day SANA and semaglutide (2 nmol/kg/day).

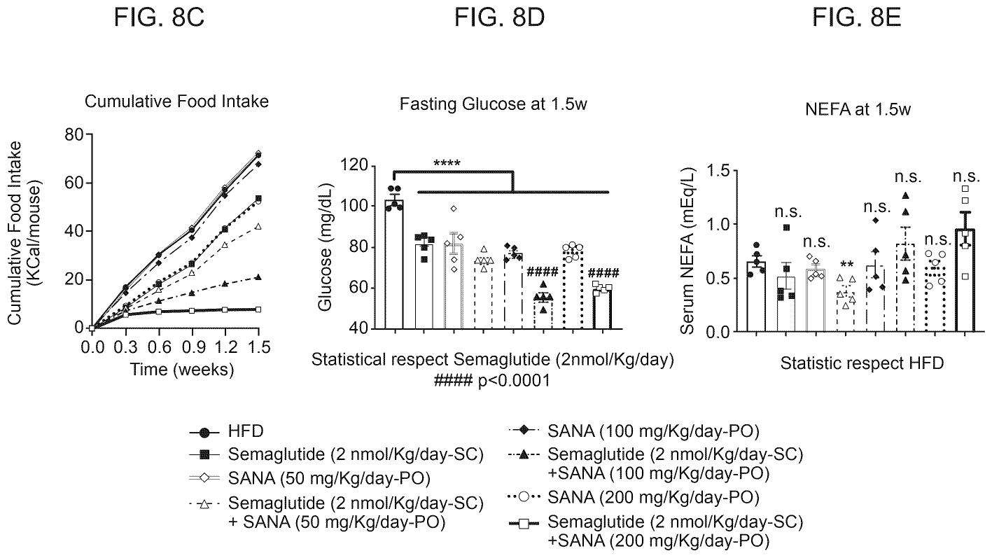

Referring to FIGS. 8A and 8B, obese mice administered a combination of 100 mg/kg/day SANA and 2 nmol/kg/day semaglutide or a combination of 200 mg/kg/day SANA and 2 nmol/kg/day semaglutide lost significantly more weight (30 and 35% decrease, respectively) than those receiving other treatments (10% decrease or less) or no treatment (5% increase).

Further, as shown in FIGS. 8C, 8D, and 8E, cumulative food intake and fasting glucose at 1.5 weeks were also significantly lower in groups treated a combination of 100 mg/kg/day SANA and 2 nmol/kg/day semaglutide or a combination of 200 mg/kg/day SANA and 2 nmol/kg/day semaglutide (roughly 42%-48% lower, respectively, versus the untreated control group). Finally, groups treated a combination of 100 mg/kg/day SANA and 2 nmol/kg/day semaglutide or a combination of 200 mg/kg/day SANA and 2 nmol/kg/day semaglutide demonstrated significantly higher serum non-esterified fatty acids (NEFA) at 1.5 weeks (20% and 37% higher, respectively, compared to the untreated control group). Again, these results demonstrate the synergistic effects of the present invention.

Without wishing to be bound by theory, it is postulated that the decreased food intake observed when higher doses of SANA were combined with semaglutide promotes increased lipolysis, leading to increased serum NEFA.

In summary, combination treatment with SANA and GLP-1 agonists or analogs is shown to have benefits in terms of weight reduction, improved basal glucose levels and glucose tolerance compared to treatment with SANA or GLP-1 agonists/analogs alone. Additionally SANA is shown to be an effective a replacement therapy after GLP-1 agonist/analog therapy. Furthermore, combination treatment with SANA and GLP-1 agonists/analogs is shown to have reduced weight regain effects after the reduction or halting of therapy.

What is claimed is:

1.-31. (canceled)

32. A method of treating a disease or disorder in a subject in need thereof, comprising:
administering to the subject a therapeutically effective amount of compound (I) 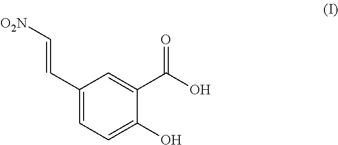 [SMILES: O=C(O)c1cc(/C=C/[N+](=O)[O-])ccc1O]
or a pharmaceutically acceptable salt thereof; and
administering to the subject a therapeutically effective amount of a GLP-1 agonist or analog,
wherein the disease or disorder is selected from obesity, type 2 diabetes, MASH (Metabolic dysfunction-associated steatohepatitis), non-alcoholic fatty liver disease, metabolic dysfunction-associated fatty liver disease, non-alcoholic steatohepatitis, and/or fibrosing non-alcoholic steatohepatitis or a combination thereof in a mammal.

33. The method of claim 32, wherein the GLP-1 agonist or analog is selected from: albiglutide (Tanzeum), aleniglipron (GSBR-1290), AMG133 (maridebart cafraglutide, MariTide), amycretin, AP025, AP026, ARI-2255, ARI-2651, bamadutide (SAR425899), beinaglutide, BGM0504 (BGM-0504, BGM 0504), BI-456906 (survodutide), bimagrumab, cagrilintide, CagriSema (cagrilintide+semaglutide), CAM-2056, cinchonine, cotadutide, CT-388 (RG6640), CT-868 (RG6641), CT-996 (RG6652), CVX-096, DA-15864, DA-3091 (microsphere formulation of exenatide), danuglipron (PF-06882961), dapiglutide (ZP7570), DD-01, DR10624, dulaglutide (Trulicity), ECC5004, ecnoglutide (XW003 (Injectable), XW004 (Oral)), efinopegdutide (HM12525A, MK-6024), efocipegtrutide (HM15211), efpeglenatide (LAPSExd4 analog, HM11260C), exenatide (Byetta, Bydureon/Bydurcon BCise), Exendin-4, GL0034 (utreglutide), GLP-1, GLP-1 eligen, Glucagon, GMA106, GRMD-0901 (ORMD-0901), GSBR-1290 (aleniglipron), GSK-2374697, HEC88473, HM15275, HRS-9531, HS-20094, HZ010, HZ012, ID110521156, Injectable HDV GLP1, Insulin glargine and lixisenatide (soliqua 100/33, LixiLan), JY09, langlenatide, LAPSGlucagon Combo (HM14320), 1Liraglutide (Victoza, Saxenda), lixisenatide (Adlyxin (US), Lyxumia (EU)), lotiglipron (PF-07081532), loxenatide, LY-2189265, LY-3305677, MAR-701, MAR709, mazdutide (IBI362, OXM3), MDR-001, MET-097i, MK-8521, MKC-253, MOD-6030, MOD-6031, noiiglutide (SHR20004, HS 20004), NN9423, NN-9709, NN-9924 (Oral semaglutide), NN-9926, NN-9277, NNC0090-2746, NNC0487-0111, NNC0519-0130, OPK88003, Oral HDV GLP1, orforglipron (LY3502970), ORMD-0901 (GRMD-0901), OWL-833, oxyntomodulin, PB-1023, PB-718, pegapamodutide, pemvidutide (ALT-801), petrelintide, PF-07081532 (lotiglipron), PF-07976016, PYY 1875 (NNC0165-1875), retatrutide (LY3437943, GGG Tri-agonist), rExendin-4, RGT-075, SAR425899 (bamadutide), SAR441255, SCO-094, semaglutide (Ozempic, Rybelsus, Wegovy), Septerna GLP-1R/GIPR/GCGR, SHR-1816, survodutide (BI-456906), taspoglutide, TB001, TG103, tirzepatide (Mounjaro, Zepbound), TT-401, TTP-054, TTP-273, UBT251, Viador-GLP-1, VK2735, vurolenatide, XW014, ZP-2929, ZP-3022, ZP-DI-70, ZP5750, ZYD-1, ZYOG-1, polymer-bound analogs thereof, pegylated analogs thereof, pharmaceutically acceptable salts thereof, and combinations thereof.

34. The method of claim 32, wherein the GLP-1 agonist or analog is selected from semaglutide, liraglutide, and pharmaceutically acceptable salts thereof.

35. The method of claim 32, wherein the therapeutically effective amount of compound (I) or pharmaceutically acceptable salt thereof is about 0.1 mg to about 400 mg, and the therapeutically effective amount of the GLP-1 agonist or analog is about 0.25 mg to about 2.4 mg.

36. The method of claim 32, wherein the therapeutically effective amount of compound (I) or pharmaceutically acceptable salt thereof is about 0.1 mg to about 400 mg, and the therapeutically effective amount of the GLP-1 agonist or analog is about 3 mg to about 14 mg.

37. The method of claim 32, wherein the therapeutically effective amount of compound (I) or pharmaceutically acceptable salt thereof is about 0.1 mg to about 400 mg, the therapeutically effective amount of the GLP-1 agonist or analog is about 0.6 mg to about 1.8 mg.

38. The method of any of claim 32, wherein the subject in need thereof undergoes a reduction in body weight within 30 days of beginning treatment.

39. The method of any of claim 32, wherein the disease or disorder is obesity.

40. The method of claim 32, wherein the subject in need thereof is a subject being administered a therapeutically effective amount of GLP-1 agonist or analog, the method comprising:
determining the subject's current dose of GLP-1 agonist or analog;
administering to the subject a therapeutically effective amount of compound (I) 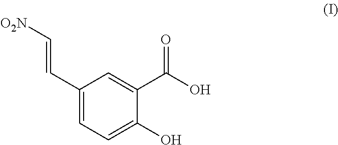 [SMILES: O=C(O)c1cc(/C=C/[N+](=O)[O-])ccc1O]
or a pharmaceutically acceptable salt thereof; and
administering to the subject a lower dose of the GLP-1 agonist or analog.

41. The method of claim 32, wherein at least one side effect or adverse effect associated with the GLP-1 agonist or analog is reduced in frequency, severity, or a combination thereof.

42. The method of claim 32, wherein the lower dose of the GLP-1 agonist or analog is at least 30% lower than the current dose, preferably at least 40% lower than the current dose, and most preferably at least 50% lower than the current dose.

43. The method of claim 32, wherein the GLP-1 agonist or analog is selected from tirzepatide, orforglipron, and pharmaceutically acceptable salts thereof.

44. A method of reducing body weight, body fat, or a combination thereof in a subject in need thereof, the method comprising:
administering to the subject a therapeutically effective amount of a GLP-1 agonist or analog; and
administering to the subject a therapeutically effective amount of compound (I) 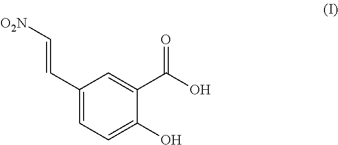 [SMILES: O=C(O)c1cc(/C=C/[N+](=O)[O-])ccc1O]
or a pharmaceutically acceptable salt thereof.

45. The method of claim 44, further comprising maintaining body weight, body fat, or a combination thereof in a subject in need thereof, the method further comprising:
administering to the subject a therapeutically effective amount of a GLP-1 agonist or analog; and
administering to the subject a therapeutically effective amount of compound (I) or a pharmaceutically acceptable salt thereof.

46. The method of claim 44, wherein the GLP-1 agonist or analog is selected from:
albiglutide (Tanzeum), aleniglipron (GSBR-1290), AMG133 (maridebart cafraglutide, MariTide), amycretin, AP025, AP026, ARI-2255, ARI-2651, bamadutide (SAR425899), beinaglutide, BGM0504 (BGM-0504, BGM 0504), BI-456906 (survodutide), bimagrumab, cagrilintide, CagriSema (cagrilintide+semaglutide), CAM-2056, cinchonine, cotadutide, CT-388 (RG6640), CT-868 (RG6641), CT-996 (RG6652), CVX-096, DA-15864, DA-3091 (microsphere formulation of exenatide), danuglipron (PF-06882961), dapiglutide (ZP7570), DD-01, DR10624, dulaglutide (Trulicity), ECC5004, ecnoglutide (XW003 (Injectable), XW004 (Oral)), efinopegdutide (HM12525A, MK-6024), efocipegtrutide (HM15211), efpeglenatide (LAPS Exd4 analog, HM11260C), exenatide (Byetta, Bydureon/Bydureon BCise), Exendin-4, GL0034 (utreglutide), GLP-1, GLP-1 eligen, Glucagon, GMA106, GRMD-0901 (ORMD-0901), GSBR-1290 (aleniglipron), GSK-2374697, HEC88473, HM15275, HRS-9531, HS-20094, HZ010, HZ012, ID110521156, Injectable HDV GLP1, Insulin glargine and lixisenatide (soliqua 100/33, LixiLan), JY09, langlenatide, LAPSGlucagon Combo (HM14320), 1Liraglutide (Victoza, Saxenda), lixisenatide (Adlyxin (US), Lyxumia (EU)), lotiglipron (PF-07081532), loxenatide, LY-2189265, LY-3305677, MAR-701, MAR709, mazdutide (IBI362, OXM3), MDR-001, MET-097i, MK-8521, MKC-253, MOD-6030, MOD-6031, noiiglutide (SHR20004, HS 20004), NN9423, NN-9709, NN-9924 (Oral semaglutide), NN-9926, NN-9277, NNC0090-2746, NNC0487-0111, NNC0519-0130, OPK88003, Oral I-DV GLP1, orforglipron (LY3502970), ORMD-0901 (GRMD-0901), OWL-833, oxyntomodulin, PB-1023, PB-718, pegapamodutide, pemvidutide (ALT-801), petrelintide, PF-07081532 (lotiglipron), PF-07976016, PYY 1875 (NNC0165-1875), retatrutide (LY3437943, GGG Tri-agonist), rExendin-4, RGT-075, SAR425899 (bamadutide), SAR441255, SCO-094, semaglutide (Ozempic, Rybelsus, Wegovy), Septerna GLP-1R/GIPR/GCGR, SHR-1816, survodutide (BI-456906), taspoglutide, TB001, TG103, tirzepatide (Mounjaro, Zepbound), TT-401, TTP-054, TTP-273, UBT251, Viador-GLP-1, V1K2735, vurolenatide, XW014, ZP-2929, ZP-3022, ZP-DI-70, ZP5750, ZYD-1, ZYOG-1, polymer-bound analogs thereof, pegylated analogs thereof, pharmaceutically acceptable salts thereof, and combinations thereof.

47. The method of claim 44 wherein the GLP-1 agonist or analog is selected from semaglutide, liraglutide, and pharmaceutically acceptable salts thereof.

48. The method of claim 44, wherein the subject in need thereof experiences a reduction in body weight within the first 30 days of administering the therapeutically effective amount of compound (I) or a pharmaceutically acceptable salt thereof.

49. The method of claim 44, wherein the GLP-1 agonist or analog is selected from tirzepatide, orforglipron, and pharmaceutically acceptable salts thereof.

50. A method of preventing weight gain in a subject currently taking a GLP-1 agonist or analog comprising:
administering to the subject a therapeutically effective amount of compound (I) 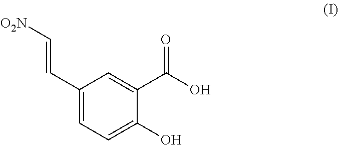 [SMILES: O=C(O)c1cc(/C=C/[N+](=O)[O-])ccc1O]
or a pharmaceutically acceptable salt thereof to the subject.

51. The method of claim 50, further comprising discontinuing the GLP-1 agonist or analog.

52. The method of claim 45, wherein the maintaining body weight, body fat, or a combination thereof in a subject in need thereof is maintaining body weight, body fat, or a combination thereof upon a reduction in dose of the GLP-1 agonist or analog, the method further comprising:
administering to the subject a therapeutically effective amount of a GLP-1 agonist or analog at a reduced dose; and
administering to the subject a therapeutically effective amount of compound (I) or a pharmaceutically acceptable salt thereof.

53. The method of claim 50 wherein the preventing of weight gain in a subject currently taking a GLP-1 agonist or analog occurs upon a reduction in dose of the GLP-1 agonist or analog.
